(19) United States
(12) Patent Application Publication
(10) Pub. No.: US 2013/0078287 A1
(43) Pub. Date: Mar. 28, 2013

(54) PHARMACEUTICAL COMPOSITIONS OF IBUPROFEN AND AN H2 RECEPTOR ANTAGONIST

(71) Applicant: Horizon Pharma USA, Inc., Deerfield, IL (US)

(72) Inventors: George F. Tidmarsh, Portola Valley, CA (US); lain Duncan, Seattle, WA (US)

(73) Assignee: Horizon Pharma USA, Inc., Deerfield, IL (US)

(21) Appl. No.: 13/681,275

(22) Filed: Nov. 19, 2012

Related U.S. Application Data
(63) Continuation of application No. PCT/US2011/038622, filed on May 31, 2011.
(60) Provisional application No. 61/350,351, filed on Jun. 1, 2010.
(60) Provisional application No. 61/351,594, filed on Jun. 4, 2010.

(51) Int. Cl. A61K 31/192; A61K 31/426; A61M 35/00; A61K 9/00

(52) U.S. Cl. CPC A61K 31/192; A61K 9/00; A61K 31/426; A61M 35/00
USPC 424/400; 514/370; 604/304

(57) ABSTRACT

Pharmaceutical compositions of a H₂ receptor antagonist and ibuprofen are provided herein. The compositions comprise, e.g., a core and a shell separated by a barrier layer, bilayered or trilayered compositions, or liquid formulations. Also provided are methods of making the pharmaceutical compositions, and methods of treatment comprising administering the pharmaceutical compositions. Also provided is a method for administration of ibuprofen to a subject in need of ibuprofen treatment is provided, in which a pharmaceutical composition comprising a therapeutically effective amount of ibuprofen and a therapeutically effective amount of an H₂RA, such as famotidine, is administered three times per day

This application claims priority to U.S. provisional application No. 61/351,594 filed on Jun. 4, 2010 and to U.S. provisional application No. 61/350,351, filed on Jun. 1, 2010, each of which is incorporated herein by reference.

Ibuprofen, a non-steroidal anti-inflammatory drug (NSAID), has been used in humans for nearly forty years. While generally regarded as safe, ibuprofen and other NSAIDs can cause gastritis, dyspepsia, and gastric and duodenal ulceration. Gastric and duodenal ulceration is a consequence of impaired mucosal integrity resulting from ibuprofen-mediated inhibition of prostaglandin synthesis. That side-effect is a particular problem for individuals who take ibuprofen for extended periods of time, such as subjects suffering from rheumatoid arthritis and osteoarthritis.

The risk of developing gastric or duodenal ulceration can be reduced by cotherapy an H₂ receptor antagonist, such as famotidine. H₂ receptor antagonists block the action of the histamine type 2 (H₂) receptor, leading to a reduction of acid secretion in the stomach.

Provided is a pharmaceutical composition comprising:

a first compartment comprising
a therapeutically effective amount of an H₂ receptor antagonist;
from about 42 mg to about 46 mg of microcrystalline cellulose;
from about 10 mg to about 19 mg of at least one binder other than microcrystalline cellulose; and
from about 0.9 mg to about 1.9 mg of at least one lubricant.

a second compartment comprising
from about 100 mg to about 850 mg of ibuprofen;
from about 200 to about 250 mg of at least one binder; and
from about 2.5 mg to about 3.5 mg of at least one lubricant.

wherein said first compartment is separated from said second compartment.

Provided is a process for preparing a pharmaceutical composition comprising an ibuprofen shell completely surrounding a coated core tablet wherein the coated core tablet comprises famotidine and a barrier layer, wherein said process comprises

blending a therapeutically effective amount of famotidine with at least one pharmaceutically acceptable excipient to yield a blended famotidine mixture;

pressing said blended famotidine mixture;

coating said pressed blended famotidine mixture with a barrier layer to yield a coated core tablet;

blending a therapeutically effective amount of ibuprofen with at least one pharmaceutically acceptable excipient to yield a blended ibuprofen mixture;

granulating said blended ibuprofen mixture to yield a granulated ibuprofen; and

compressing said granulated ibuprofen around the coated core tablet to yield an ibuprofen shell such that the ibuprofen shell completely surrounds the coated core tablet.

Also provided is a pharmaceutical composition prepared by a process described herein.

Also provided is a method for reducing the risk of developing ibuprofen-induced ulcers in a human subject requiring ibuprofen for an ibuprofen-responsive condition comprising administering to the human subject a pharmaceutical composition described herein.

Also provided is a method of treating a subject in need of ibuprofen and an H₂RA treatment comprising prescribing or administering to the subject a pharmaceutical composition described herein.

Also provided is a method for reducing the incidence of ibuprofen-induced gastric and/or duodenal ulcers in a subject in need of ibuprofen comprising prescribing or administering to the subject a pharmaceutical composition described herein.

Also provided is a method for reducing gastric acid while treating a subject with an ibuprofen-responsive condition comprising prescribing or administering to the subject a pharmaceutical composition described herein.

Also provided is a method of reducing or preventing the occurrence of gastrointestinal toxicity associated with the use of ibuprofen comprising prescribing or administering a pharmaceutical compositions described herein.

Also provided is a method of reducing symptoms of a famotidine-responsive condition in a subject in need of NSAID treatment who has experienced symptoms of a famotidine-responsive condition associated with NSAID administration, comprising prescribing or administering a pharmaceutical compositions described herein.

Also provided is a method for preventing toxicities associated with ibuprofen use in a subject who is at risk for the development of such toxicities comprising prescribing or administering a pharmaceutical composition described herein.

Also provided is a method for reducing the risk of an adverse event in an subject requiring ibuprofen for an ibuprofen-responsive comprising:

a) determining an approximate serum creatinine concentration for the individual;

b) if the subject has a creatinine clearance rate of greater than about 50 mL/minute, then prescribing or administering a first dose of a pharmaceutical composition described herein,

c) prescribing or administering to the human subject a second dose of the pharmaceutical composition; and

d) prescribing or administering to the human subject a third dose of the pharmaceutical composition.

Also provided is a method for the treatment of cystic fibrosis comprising prescribing or administering a pharmaceutical composition described herein.

### BRIEF DESCRIPTION OF THE FIGURES

FIG. 1 shows the incidence rate of endoscopic gastric ulcers in the REDUCE-1 study (life table and crude rate).

FIG. 2 shows the incidence rate of endoscopic upper GI such as gastric and/or duodenal ulcers in the REDUCE-2 study (life table and crude rate).

FIG. 3 shows the incidence rate of endoscopic UGI ulcers in REDUCE-1 (R1) and REDUCE-2 (R2).

FIG. 4 shows the integrated results of REDUCE-1 and REDUCE-2 studies with respect to the incidence of UGI ulcers.

FIG. 5 shows the incidence rate (life table) of endoscopic upper GI such as gastric and/or duodenal ulcers in the REDUCE-1 study.

FIG. 6 shows the incidence rate (crude rate) of endoscopic upper GI (gastric and/or duodenal) ulcers in the REDUCE-1 study.

FIG. 7 shows the incidence rate (life table) of endoscopic gastric ulcers in the REDUCE-2 study.

FIG. 8 shows the incidence rate (crude rate) of endoscopic gastric ulcers in the REDUCE-2 study.

FIG. 9 shows the incidence rate (life table) of endoscopic gastric and duodenal ulcers in the REDUCE-1 study.

FIG. 10 shows the incidence rate of endoscopic gastric and duodenal ulcers in the REDUCE-2 study (crude rate).

FIG. 11 shows the incidence by time of gastric and duodenal ulcers in the REDUCE-1 study.

FIG. 12 shows the incidence by time of gastric and duodenal ulcers in the REDUCE-1 study.

FIG. 13 shows the incidence rate of endoscopic gastric ulcers in subjects with/without low dose aspirin and/or OAC in the REDUCE-1 study.

FIG. 14 shows the incidence rate of endoscopic gastric ulcers in subjects with/without low dose aspirin and/or OAC in the REDUCE-2 study.

FIG. 15 shows the percentage of subjects who completed study in the REDUCE-1 study.

FIG. 16 shows the percentage of subjects who completed study in the REDUCE-2 study.

FIG. 17 shows the discontinuation rates due to gastrointestinal adverse events for HZT-501 and Ibuprofen in the REDUCE-1 study.

FIG. 18 shows the discontinuation rates due to gastrointestinal adverse events for HZT-501 and Ibuprofen in the REDUCE-2 study.

FIG. 19 shows the forest plot of the relative risks (95% CI) of upper gastrointestinal ulcers for HZT-501 v. ibuprofen in subgroup analyses.

FIG. 20 shows the percentage of subjects who completed the follow-on study.

FIG. 21 shows the percentage of subjects who withdraw from the follow-on study due to adverse events.

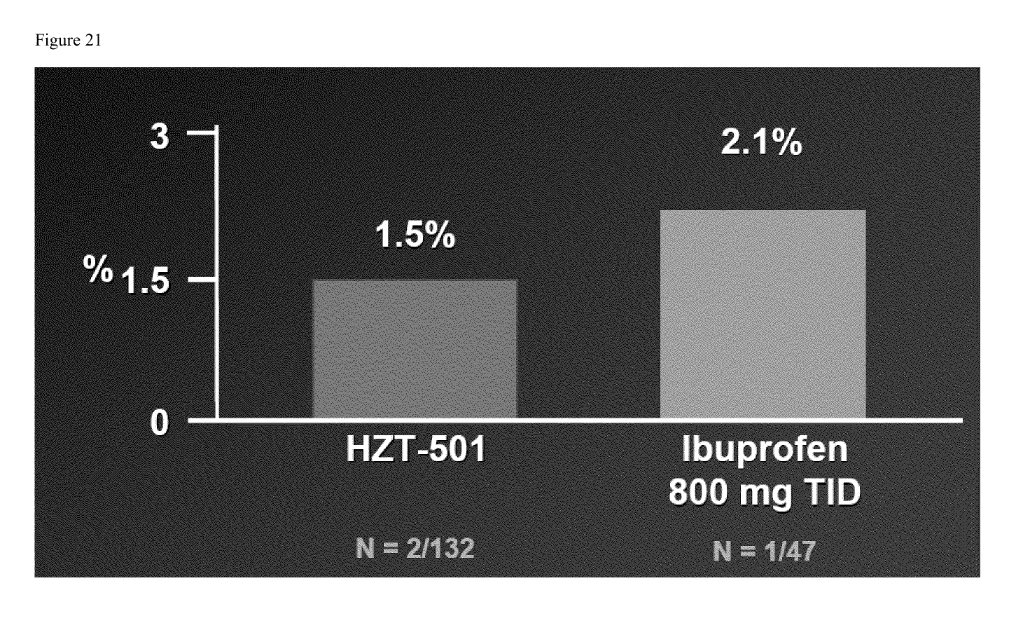

As used in the present specification, the following words and phrases are generally intended to have the meanings as set forth below, except to the extent that the context in which they are used indicates otherwise.

“H₂ receptor antagonists” refer to a class of drugs that are capable of blocking the action of histamine on parietal cells in the stomach and decreasing the production of acid by these cells. Examples of H₂ receptor antagonists include without limitation, cimetidine, famotidine, nizatidine, and ranitidine. In some embodiments, the H₂ receptor antagonist is famotidine. In some embodiments, the H₂ receptor antagonist is ranitidine.

“Famotidine” refers to 3-[2-(diaminomethyleneamino)thiazol-4-ylmethylthio]-N-sulfamoylpropionamidine, as well as pharmaceutically acceptable salts thereof. Famotidine also is intended to encompass all known polymorphic forms, including without limitation the amorphous form, polymorphic Form A, Form B, or Form C and their mixtures. Famotidine can be prepared using art-known methods. Famotidine's properties have been described in the medical literature.

“Ranitidine” refers to N-(2-[(5-(dimethylaminomethyl)furan-2-yl)methylthio]ethyl)-N-methyl-2-nitroethene-1,1-diamine, and pharmaceutically acceptable salts thereof. Ranitidine is intended to include all known polymorphic forms including form I and form II of the hydrochloric acid salt of ranitidine, and mixtures thereof.

“Ibuprofen” refers to 2-(p-isobutylphenyl)propionic acid (C₁₃H₁₈O₂), including various polymorphic forms and pharmaceutically acceptable salts. Two enantiomers of ibuprofen exist. As used herein in the context of the pharmaceutical compositions described herein, “ibuprofen” refers to a racemic mixture of both enantiomers, as well as mixtures that contain more of one enantiomer than another (including, for example, mixtures enriched in the S-enantiomer), and enantiomerically pure preparations (including, for example, the S-enantiomer substantially free of the R-enantiomer). Ibuprofen is available commercially, typically as a racemic mixture, and, for example, ibuprofen with mean particle sizes of 25, 38, 50, or 90 microns can be obtained from BASF Aktiengesellschaft (Ludwigshafen, Germany). One ibuprofen product is a directly compressible formulation described in WO 2007/042445, a version of which is available from BASF under the trade name Ibuprofen DC 85. Ibuprofen DC 85 is a roller-compacted granulation comprising 85% ibuprofen, 6.6% microcrystalline cellulose, 5.4% colloidal silicon dioxide, and 2.9% croscarmellose sodium. Ibuprofen is also available from Albemarle Corporation and other companies. Ibuprofen's properties have been described in the medical literature.

A “therapeutically effective amount” of ibuprofen is an amount of ibuprofen or its pharmaceutically acceptable salt which eliminates, alleviates, or provides relief of the symptoms for which it is administered.

A “therapeutically effective amount” of the H₂ receptor antagonist, such as famotidine, is an amount of H₂ receptor antagonist which suppresses gastric acid secretion, or otherwise eliminates, alleviates, or provides relief of the symptoms for which it is administered.

A “compartment” is a physical region, e.g., of a tablet or other dosage form. Two components are distinct compartments if there exists a delineation between the two components, even though they may be in direct physical contact with one another. The delineation may or may not be visible to the naked eye, and may be observed using X-rays or other methods.

The term “core,” as used herein, refers to a interior compartment of a unit dosage form. In some embodiments, the core is a single interior compartment. In some embodiments, the core may be beads, which, e.g., can be used to form a bead containing matrix or a multiparticulate formulation. In some embodiments, the multiparticulate formulation comprises an ibuprofen matrix into which are dispersed a plurality of famotidine or another H₂ receptor antagonist beads.

The term “shell,” or “mantle,” as used herein, refers to an exterior compartment of a unit dosage form, which substantially surrounds the core. In some embodiments, the shell completely surrounds the core. As described herein, this exterior compartment may, in some embodiments, be over-coated for cosmetic or other reasons.

The term “direct physical contact” refers to the absence of a barrier layer between components or adjacent compartments of a unit dosage form.

The term “barrier layer” refers to a layer or film that is interposed between the ibuprofen-containing compartment (e.g., an ibuprofen core or coated ibuprofen particles) and the famotidine or another H₂ receptor antagonist-containing compartment (e.g., famotidine-containing coating or coated famotidine particles). Materials useful in a “barrier layer” including in a “barrier layer coating” include, without limitation, water soluble polysaccharide gums such as carrageenan, fucoidan, gum ghatti, tragacanth, arabinogalactan, pectin, and xanthan; water-soluble salts of polysaccharide gums such as sodium alginate, sodium tragacanthin, and sodium gum ghattate; water-soluble hydroxyalkylcellulose wherein the alkyl member is straight or branched of 1 to 7 carbons such as, for example, hydroxymethylcellulose, hydroxyethylcellulose, and hydroxypropylcellulose; synthetic water-soluble cellulose-based lamina formers such as, for example, methyl cellulose and its hydroxyalkyl methylcellulose cellulose derivatives such as a member chosen from the group of hydroxyethyl methylcellulose, hydroxypropyl methyl cellulose, and hydroxybutyl methylcellulose; croscarmellose sodium; and other cellulose polymers such as sodium carboxymethylcellulose. Other lamina forming materials that can be used for this purpose include, for example, poly(vinylpyrrolidone), polyvinylalcohol, polyethylene oxide, a blend of gelatin and polyvinyl-pyrrolidone, gelatin, glucose, saccharides, povidone, copovidone, poly(vinylpyrrolidone)-poly(vinyl acetate) copolymer.

An “excipient,” as used herein, is any component of a pharmaceutical composition that is not an active pharmaceutical ingredient (i.e., ibuprofen and/or famotidine). Excipients include binders, lubricants, disintegrants, coatings, barrier layer components, glidants, and other components. Excipients are known in the art. Some excipients serve multiple functions or are so-called high functionality excipients. For example, talc may act as a lubricant and a glidant. In some embodiments, the excipient is USP, EU, JP grade or national formulary (NF) grade.

A “binder” (also called an “adhesive”) refers to a material that can be added to impart cohesive qualities to components of a pharmaceutical composition. Non-limiting examples of binders include starch, sugars such as sucrose, glucose, dextrose and lactose, hydrogenated vegetable oil, castor oil, paraffin, higher aliphatic alcohols, higher aliphatic acids, long chain fatty acids, fatty acid esters, wax-like materials such as fatty alcohols, fatty acid esters, fatty acid glycerides, hydrogenated fats, hydrocarbons, normal waxes, stearic acid, stearyl alcohol, hydrophobic and hydrophilic polymers having hydrocarbon backbones, and mixtures thereof. Non-limiting examples of water-soluble binders include modified starch, gelatin, polyvinylpyrrolidone, cellulose derivatives such as powdered cellulose, microcrystalline cellulose, silicified microcrystalline cellulose (SMCC), hydroxypropylcellulose, low-substituted hydroxypropylcellulose, hypromellose (hydroxypropylmethylcellulose), polyvinyl alcohol and mixtures thereof.

“Disintegrants” refer to excipients useful in ensuring that the pharmaceutical composition has an acceptable disintegration rate in an environment of use. Examples of disintegrants include croscarmellose sodium, starch derivatives (e.g., sodium carboxymethyl starch and pregelatinized corn starch such as starch 1500 from Colorcon) and salts of carboxymethylcellulose (e.g., sodium carboxymethylcellulose), crospovidone (cross-linked PVP polyvinylpyrrolidinone (PVP), e.g., Polyplasdone™ from ISP or Kollidon™ from BASF).

“Glidants” refer to excipients included in a pharmaceutical composition to keep the component powder flowing as a tablet is being made, preventing formation of lumps. Nonlimiting examples of glidants are colloidal silicon dioxides such as CAB-O-SIL™ (Cabot Corp.), SYLOID™, (W.R. Grace & Co.), AEROSIL™ (Degussa), talc, and corn starch.

A “filler” is an excipient added to increase one or more of the bulk, weight, viscosity, opacity, or strength of a composition. Examples of fillers include, without limitation, calcium phosphate dibasic, tricalcium phosphate, calcium carbonate, starch (such as corn, maize, potato and rice starches), and modified starches (such as carboxymethyl starch, etc.).

“Lubricant” refers to an excipient that reduces sticking by a solid formulation to the equipment used for production of a unit does form, such as, for example, the punches of a tablet press. Non-limiting examples of lubricants include, without limitation, talc, glyceryl dibehenate (e.g., CompritolATO888™ Gattefosse France), stearic acid, hydrogenated vegetable oils (such as hydrogenated cottonseed oil (STEROTEX™), hydrogenated soybean oil (STEROTEX™ HM) and hydrogenated soybean oil & castor wax (STEROTEX™ K), stearyl alcohol, leucine, polyethylene glycol, aluminum stearate, magnesium stearate, calcium stearate, glyceryl monostearate, stearic acid, polyethylene glycol, polyoxyethylene glycol (PEG, BASF) such as PEG 4000-8000, glyceryl mono fatty acid (e.g., glyceryl monostearate from Danisco, UK), glyceryl palmito-stearic ester (e.g., Precirol™, Gattefosse France), sodium benzoate, sodium lauryl sulfate, magnesium lauryl sulfate, sodium oleate, sodium stearyl fumarate, DL-leucine, and colloidal silica.

A “plasticizer” refers to an excipient that can impart flexibility and/or stretchability to a coat or membrane. Examples of plasticizers include, without limitation, phthalates, phosphates, citrates, adipates, tartrates, sebacates, succinates, glycolates, glycerolates, benzoates, myristates, sulfonamides halogenated phenyls, poly(alkylene glycols), poly(alkylenediols), polyesters of alkylene glycols, alkyl citrate and citrates esters such as tributyl citrate, triethyl citrate, and acetyl triethyl citrate; alkyl adipates, such as for example, dioctyl adipate, diethyl adipate and di(2-methoxyethyl)adipate; dialkyl tartrates, such as for example, diethyl tartrates and dibutyl tartrate; alkyl sebacates, such as for example, diethyl sebacate, dipropyl sebacate and dinonyl sebacate; alkyl succinates, such as for example, diethyl succinate and dibutyl succinate; alkyl glycolates, alkyl glycerolates, glycol esters and glycerol esters, such as for example, glycerol diacetate, glycerol triacetate, glycerol monolactate diacetate, ethylene glycol diacetate, ethylene glycol dibutyrate, triethylene glycol diacetate, triethylene glycol dibutyrate, triethylene glycol dipropionate and mixtures thereof. Other plasticizers include camphor, N-ethyl (o- and p-toulene)sulfonamide, N-cyclohexyl-p-toluene sulfonamide, substituted epoxides and mixtures thereof.

A “surfactant” refers to one or more excipients that may be added to the pharmaceutical composition to facilitate dissolution of poorly soluble excipients and/or to increase dissolution rate of pharmaceutical composition or components thereof. Surfactants include hydrophilic surfactants or lipophilic surfactants or mixtures thereof. The surfactants can be anionic, nonionic, cationic, and zwitterionic surfactants. The hydrophilic non-ionic surfactants include, without limitation, polyethylene glycol sorbitan fatty acid esters, tocopheryl polyethylene glycol 1000 succinate, and hydrophilic transesterification products of at least one member of the group from triglycerides, vegetable oils, and hydrogenated vegetable oils with a polyol such as glycerol, polyethylene glycol, sorbitol, pentaerythritol, or a saccharide. The ionic surfactants include without limitation, alkylammonium salts, fusidic acid salts, fatty acid derivatives of amino acids, oligopeptides, and polypeptides, glyceride derivatives of amino acids, lecithins and hydrogenated lecithins, lysolecithins and hydrogenated lysolecithins, phospholipids and derivatives thereof, lysophospholipids and derivatives thereof, carnitine fatty acid ester salts, salts of alkylsulfates, fatty acid salts, sodium docusate, acyl lactylates, mono- and di-acetylated tartaric acid esters of mono- and di-glycerides, succinylated mono- and di-glycerides, citric acid esters of mono- and di-glycerides, and mixtures thereof. The lipophilic surfactants include without limitation, fatty alcohols, glycerol fatty acid esters, acetylated glycerol fatty acid esters, lower alcohol fatty acids esters, propylene glycol fatty acid esters, sorbitan fatty acid esters, polyethylene glycol sorbitan fatty acid esters, sterols and sterol derivatives, polyoxyethylated sterols and sterol derivatives, polyethylene glycol alkyl ethers, sugar esters, sugar ethers, lactic acid derivatives of mono- and di-glycerides, hydrophobic transesterification products of a polyol with at least one member of the group from glycerides, vegetable oils, hydrogenated vegetable oils, fatty acids and sterols, oil-soluble vitamins/vitamin derivatives, PEG sorbitan fatty acid esters, PEG glycerol fatty acid esters, polyglycerized fatty acid, polyoxyethylene-polyoxypropylene block copolymers, sorbitan fatty acid esters, and mixtures thereof. Surfactants also include, PEG-20-glyceryl stearate (CAPMUL™), PEG-40 hydrogenated castor oil (CREMOPHOR RH 40™), PEG 6 corn oil (LABRAFIL™), lauryl macrogol-32 glyceride (GELUCIRE44/14™) stearoyl macrogol glyceride (GELUCIRE50/13™), polyglyceryl-10 mono dioleate (CAPROL™ PEG860), propylene glycol oleate (LUTROL™), propylene glycol dioctanoate (CAPTEX), propylene glycol caprylate/caprate (LABRAFAC™), glyceryl monooleate (PECEOL™), glycerol monolinoleate (MAISINE™), glycerol monostearate (CAPMULT), PEG-20 sorbitan monolaurate (TWEEN20™), PEG-4 lauryl ether (BRIJ30™), sucrose distearate (SUCROESTER7™), sucrose monopalmitate (SUCROESTER15™), polyoxyethylene-polyoxypropylene block copolymer (LUTROL™), polyethylene glycol 660 hydroxystearate, (SOLUTOL™), sodium lauryl sulfate, sodium dodecyl sulphate, dioctyl suphosuccinate, L-hydroxypropyl cellulose, hydroxylethylcellulose, hydroxylpropylcellulose, propylene glycol alginate, sodium taurocholate, sodium glycocholate, sodium deoxycholate, betains, polyethylene glycol (CARBOWAX™), d-tocopheryl polyethylene glycol 1000 succinate, (VITAMIN E TPGS™), and mixtures thereof.

As used herein, a “pharmaceutically acceptable” component is one that is suitable for use with humans and/or animals without undue adverse side effects (such as toxicity, irritation, and allergic response) commensurate with a reasonable benefit/risk ratio.

The term “stable,” as used herein, refers to a composition in which the active pharmaceutical ingredients (e.g., ibuprofen and famotidine) are present in an amount of at least about 90%, and such as at least about 95%, at least about 96%, at least about 97%, at least about 98%, at least about 99%, or at least about 99.5% of the originally specified amount for each such ingredient after one or more time periods chosen from about 1, about 3, about 6, about 9, and about 12 months at about 25° C. and about 65% relative humidity. In some embodiments, no more than about 3%, no more than about 2%, no more than about 1%, no more than about 0.9%, no more than about 0.8%, no more than about 0.7%, or no more than about 0.6% sulfamide is present after a specified period of time and under specified conditions after one or more time periods chosen from about 1, about 3, about 6, about 9, or about 12 months at about 25° C. and about 60% relative humidity.

Creatinine clearance refers to a measure of the rate at which the kidneys filter creatinine out of the blood. Creatinine clearance can be calculated using serum creatinine concentration. The Cockcroft-Gault equation estimates creatinine clearance on the basis of serum creatinine level, age, sex and weight (see Traynor et al. BMJ 33:733-737 (2006) and references therein). It is based on creatinine excretion in men with normal renal function with a correction for women. An individual's ideal or otherwise adjusted body weight can be used in the Cockcroft-Gault equation or an individual's actual body weight can be used.

A Cockcroft-Gault equation is:

$$Estimated creatinine clearance ( {Cl}_{Cr} ) = \frac{( 140 - age ) \times weight \times 1.2}{SCr} \times ( 0.85 if female )$$

where age is expressed in years, SCr in micromole/L and weight in kg.

A Cockcroft-Gault equation using ideal body weight (IBW) is:

### Female:

$$GFR ( mL \text{/} min ) = 0.85 \times \frac{( 140 - age ) \times ideal body weight ( kg )}{72 \times serum creatinine ( mg \text{/} dL )}$$

### Male:

$$GFR ( mL \text{/} min ) = \frac{( 140 - age ) \times ideal body weight ( kg )}{72 \times serum creatinine ( mg \text{/} dL )}$$

### Estimate Ideal Body Weight (IBW) in kg

Females: IBW=45.5 kg+2.3 kg for each inch over 5 feet
Males: IBW=50 kg+2.3 kg for each inch over 5 feet

“QD”, “BID”, “TID”, “QID”, and “HS” have their usual meanings of, respectively, administration of medicine once per day, twice per day, three times per day, four times per day or at bedtime. Administration three times per day means that at least about 6 hours, such as at least about 7 hours, for example, about 8 hours elapse between administrations. Administration three times per day can mean administration about every 8 hours (e.g., 7 a.m., 3 p.m. and 11 p.m.). In some cases in which quantitative measurements are made, “TID administration” can mean administration every 8±0.25 hours.

As used herein, the term “daily quantity” refers to the quantity of an API administered over a 24-hour period under a specific dosing regimen.

A “subject in need of ibuprofen treatment” is a human individual who receives therapeutic benefit from administration of ibuprofen.

An “ibuprofen responsive condition” is a condition for which symptoms are reduced by administration of ibuprofen.

A “subject in need of famotidine treatment” is a human individual who receives therapeutic benefit from administration of famotidine.

A “famotidine responsive condition” is a condition for which symptoms are reduced by administration of famotidine.

A subject is “at elevated risk for developing an NSAID-induced ulcer” if the subject in more susceptible than the average individual to development of an ulcer when under treatment with an NSAID. A high odds ratio for risk of development of NSAID-associated ulcer complications is seen in individuals with a past complicated ulcer (odds ratio 13.5), individuals taking multiple NSAIDs or NSAIDs plus aspirin (odds ratio 9.0); individuals taking high doses of NSAIDs (odds ratio 7.0), individuals under anticoagulant therapy, such as low dose aspirin (odds ration 6.4), individuals with a past uncomplicated ulcer (odds ratio 6.1), and individuals older than 70 years (odds ratio 5.6) See, e.g., Gabriel et al., 1991, Ann Intern Med. 115:787; Garcia Rodriguez et al. 1994, Lancet 343:769; Silverstein et al. 1995, Ann Intern Med. 123:241; and Sorensen et al., 2000, Am J. Gastroenterol. 95:2218. Subjects at increased risk for developing an NSAID-induced ulcer may have one or more of these risk factors.

Subjects “at high risk for developing an NSAID-induced ulcer” are individuals older than 80 years of age and subjects with a history of NSAID-associated serious gastrointestinal complications (e.g., perforation of ulcers, gastric outlet obstruction due to ulcers, gastrointestinal bleeding).

As used herein, “dyspepsia” refers to upper abdominal pain or discomfort with or without symptoms of early satiety, nausea, or vomiting with no definable organic cause, as diagnosed following the Rome II criteria (Talley et al., 1999, Gut 45 (Suppl. II):1137-42), or any subsequent modification thereof. According to the Rome II criteria, a diagnosis of functional dyspepsia requires: (1) persistent or recurrent abdominal pain or discomfort centered in the upper abdomen; (2) symptom duration of at least 12 weeks, which need not be consecutive, within the preceding 12 months; (3) no evidence of organic disease (including at upper endoscopy) that is likely to explain symptoms; (4) no evidence that dyspepsia is exclusively relieved by defecation or association with the onset of a change in the stool frequency or stool form (i.e., not irritable bowel syndrome). In this context, “discomfort” is defined as an unpleasant sensation, and may include fullness, bloating, early satiety, and nausea. The definition includes, without limitation, ulcer-like, dysmotility-like, and non-specific dyspepsia. Symptoms of dyspepsia include nausea, regurgitation, vomiting, heartburn, prolonged abdominal fullness or bloating after a meal, stomach discomfort or pain, and early fullness.

As used herein, a person with “normal body weight” has a body mass index of 20-25 inclusive (calculated as weight (kg)/[height (m)]²).

As used herein, a “24-hour dosing cycle” or “24-hour dosing period” refers to a 24-hour period of time during which a subject is administered drug(s) and may correspond to a calender day (e.g., 12:01 a.m. to midnight) or may span two calender days (noon day 1 to noon day 2).

The term “about,” as used herein, is intended to indicate up to ±10%.

The term “substantially,” as used herein with reference to the core of a pharmaceutical composition refers to variability resulting from manufacturing tolerances, as well as intentional deviations from these precise geometric shapes. In this context, the term “substantially” is intended to indicate a tolerance for a deviation of +−5%.

All percentages are % w/w, unless specifically indicated otherwise. Unless otherwise indicated, “% weight” is percent weight of the specified component compared to the total weight of the unit dosage (e.g., tablet). Optionally the % weight can be calculated as if the total weight of the unit dosage form is the weight of the ibuprofen portion, famotidine portion, and barrier layer, but not including the over-coating (e.g., added to mask taste, improve ease of swallowing, improve appearance, and the like). Optionally the % weight can be calculated based on the total weight of the unit dosage form, including all coatings. “United States Pharmacopeia” and “USP” mean the United States Pharmacopeia and National Formulary 29th Revision (available from 12601 Twinbrook Parkway, Rockville, Md. 20852-1790, USA). It will be appreciated that due to rounding or practical limits on quantitative measurements, reference to a quantity of API or excipient in a dosage form can include some variation, such as ±10%, for example, ±5%, such as ±1%. It will be appreciated, for example, that a total quantity of 80 mg famotidine can be administered in three doses of 26.6 mg famotidine per dose.

Provided is a pharmaceutical composition comprising:

a first compartment comprising
a therapeutically effective amount of an H₂ receptor antagonist;
from about 42 mg to about 46 mg of microcrystalline cellulose;
from about 10 mg to about 19 mg of at least one binder other than microcrystalline cellulose; and
from about 0.9 mg to about 1.9 mg of at least one lubricant; and

a second compartment comprising
a therapeutically effective amount of ibuprofen;
from about 200 to about 250 mg of at least one binder; and
from about 2.5 mg to about 3.5 mg of at least one lubricant.

wherein said first compartment is separated from said second compartment.

In some embodiments, the pharmaceutical composition has a core shell architecture. Accordingly, provided is a pharmaceutical composition comprising

a coated core tablet, wherein the coated core tablet comprises
a barrier layer coating; and
a core tablet coated with the barrier layer; and

a shell completely surrounding said coated core tablet,

wherein one of the coated core tablet or the shell comprises
a therapeutically effective amount of an H₂ receptor antagonist;
from about 42 mg to about 46 mg of microcrystalline cellulose;
from about 10 mg to about 19 mg of at least one binder other than microcrystalline cellulose; and
from about 0.9 mg to about 1.9 mg of at least one lubricant; and

the other of the coated core tablet or the shell comprises
a therapeutically effective amount of ibuprofen;
from about 200 to about 250 mg of at least one binder; and
from about 2.5 mg to about 3.5 mg of at least one lubricant.

Also provided is a pharmaceutical composition comprising

a coated core tablet, wherein the coated core tablet comprises
a barrier layer coating; and
a core tablet coated with said barrier layer coating wherein said core tablet comprises
   from about 24 mg to about 28 mg famotidine;
   from about 42 mg to about 46 mg of microcrystalline cellulose;
   from about 10 mg to about 19 mg of at least one binder other than microcrystalline cellulose; and
   from about 0.9 mg to about 1.9 mg of at least one lubricant; and

an ibuprofen shell completely surrounding said coated core tablet, wherein said ibuprofen shell comprises
from about 750 mg to about 850 mg ibuprofen;
from about 200 to about 250 mg of at least one binder; and
from about 2.5 mg to about 3.5 mg of at least one lubricant.

Also provided is a pharmaceutical composition comprising

a coated core tablet, wherein the coated core tablet comprises
a barrier layer coating; and
a core tablet coated with said barrier layer wherein said core tablet comprises
   from about 13 mg to about 15 mg famotidine;
   up to about 46 mg of microcrystalline cellulose;
   up to about 19 mg of at least one binder other than microcrystalline cellulose; and
   up to about 1.9 mg of at least one lubricant; and
an ibuprofen shell completely surrounding said coated core tablet, wherein said ibuprofen shell comprises
   from about 100 mg to about 450 mg ibuprofen;
   up to about 250 mg of microcrystalline cellulose; and
   up to about 3.5 mg of at least one lubricant.

In some embodiments, the pharmaceutical composition has a bilayer architecture wherein the first compartment corresponds to a first layer of the pharmaceutical composition and the second compartment corresponds to a second layer of the pharmaceutical composition, wherein the first layer and the second layer are separated by a barrier layer. Accordingly, also provided is a pharmaceutical composition comprising

a first layer comprising a therapeutically effective amount of an H₂ receptor antagonist and

a second layer comprising a therapeutically effective amount of ibuprofen,

where the first layer and the second layer are separated by a first barrier layer.

In some embodiments, the pharmaceutical composition has a trilayer architecture. Accordingly, also provided is a pharmaceutical composition wherein the first compartment corresponds to a first layer of the pharmaceutical composition, a portion of the second compartment corresponds to a second layer of the pharmaceutical composition adjacent to a first side of said first layer, and the remainder of the second compartment corresponds to a third layer of the pharmaceutical composition adjacent to a second side of the first layer, where the first layer and the second layer are separated by a first barrier layer, and the first layer and the third layer are separated by a second barrier layer. Also provided is a pharmaceutical composition comprising a first layer comprising

a first layer comprising a therapeutically effective amount of an H₂ receptor antagonist,

a second layer comprising ibuprofen, and

a third layer comprising ibuprofen,

wherein the first layer is adjacent to a first side of the first layer, and the third layer is adjacent to a second side of the first layer,

wherein the total amount of ibuprofen in the pharmaceutical composition is a therapeutically effective amount, and

wherein the first layer and the second layer are separated by a first barrier layer, and the first layer and the third layer are separated by a second barrier layer.

In some embodiments, the first barrier layer is the same, both in amount and content, as the second barrier layer. In some embodiments, the first barrier layer is different, either in amount and/or content, from the second barrier layer.

In some embodiments of the pharmaceutical composition having a bilayer and/or trilayer architecture, the first compartment may include larger amount of excipients, compared to the amounts of excipients used in the core-shell or tablet-in-tablet pharmaceutical compositions disclosed here. In some embodiments, the amount of excipients used is 2-10 times more than that used in the core-shell or tablet-in-tablet pharmaceutical compositions disclosed here. In some embodiments, the amount of excipients used is about 700 mg or more.

In some embodiments, the pharmaceutical composition is in the form of a soft gel capsule. Accordingly, also provided is a pharmaceutical composition comprising:

a soft gel capsule containing therein a first compartment comprising a therapeutically effective amount of an H₂ receptor antagonist, and

a second compartment comprising a therapeutically effective amount of ibuprofen,

wherein the first compartment is separated from the second compartment.

In some embodiments, the soft gel capsule comprises gelatin. In some embodiments, the soft gel capsule comprises gelatin, water, an opacifier, and a plasticizer, such as glycerin and/or sorbitol(s). In some embodiments, the soft gel capsule is commercially available from Catalent Pharma Solutions.

In some embodiments, first compartment further comprises a nonaqueous liquid such as an oil wherein the an H₂ receptor antagonist is dissolved or suspended in the nonaqueous liquid. In some embodiments, second compartment further comprises a nonaqueous liquid such as an oil wherein the ibuprofen is dissolved or suspended in the nonaqueous liquid.

In some embodiments, the pharmaceutical composition is in the form of a hard gel capsule. Accordingly, also provided is a pharmaceutical composition comprising:

a hard gel capsule containing therein a first compartment comprising a therapeutically effective amount of an H₂ receptor antagonist, and a second compartment comprising a therapeutically effective amount of ibuprofen,

wherein the first compartment is separated from the second compartment.

In some embodiments, the second compartment comprises powdered ibuprofen optionally with one or more excipients.

In some embodiments, the first compartment comprises a core of an H₂ receptor antagonist. In some embodiments, the H₂ receptor antagonist is present as multiple particles. In some embodiments, the H₂ receptor antagonist is present as multiple particles and are blended or otherwise mixed with powdered ibuprofen powder, optionally with one or more excipients. In some embodiments, the H₂ receptor antagonist is present as multiple particles and are blended or otherwise mixed with ibuprofen granules or particles.

In some embodiments, the pharmaceutical composition is in a chewable form. Accordingly, also provided is a pharmaceutical composition comprising:

a first compartment comprising a therapeutically effective amount of an H₂ receptor antagonist, and

a second compartment comprising a therapeutically effective amount of ibuprofen, and

further comprising a binding agent and a sweetener,

wherein the first compartment is separated from the second compartment, and wherein the pharmaceutical composition is in a chewable form.

In some embodiments, the binding agent is chosen from pectin, gelatin, starch, and mixtures thereof. Chewable forms for delivering pharmaceutical agents and methods for making such forms are well know in the art. See, e.g., US Patent Publication No. 2010/0330058 which is incorporated herein by reference.

In some embodiments, the pharmaceutical composition is in a form that dissolves and/or disintegrates orally. Accordingly, also provided is a pharmaceutical composition comprising:

a first compartment comprising a therapeutically effective amount of an H₂ receptor antagonist, and

a second compartment comprising a therapeutically effective amount of ibuprofen,

wherein the first compartment is separated from the second compartment, and

wherein the first compartment and the second compartment are present in an orally dissolving film.

In some embodiments, the pharmaceutically composition comprises a single layer of orally dissolvable film. In some embodiments, the pharmaceutically composition comprises multiple layers of orally dissolvable film. In some embodiments, pharmaceutically composition comprises a first layer of orally dissolvable film comprising a therapeutically effective amount of ibuprofen and a second layer of orally dissolvable film comprising a therapeutically effective amount of an H₂ receptor antagonist. Orally dissolving films and methods for making such films are well know in the art. See, e.g., US Patent Publication No. 2010/0227854 which is incorporated herein by reference.

In some embodiments, the pharmaceutical composition is in a tablet form, including without limitation, a core-shell architecture, a bilayer tablet, or a trilayer tablet, and the pharmaceutical composition further comprises a coating agent surrounding the ibuprofen shell to yield a coated pharmaceutical composition. In some embodiments, the coating agent serves to improve appearance, taste, or swallowability of the pharmaceutical composition. In some embodiments, the coating agent is comprises a mixtures of polymers, plasticizers, coloring agents and other excipients. In some embodiments, the coating agent can be stirred into water or an organic solvent to produce a dispersion for the film coating of solid oral dosage forms such as tablets. In some embodiments, a readily soluble film is used. Materials that can be used for readily soluble films include cellulose derivatives (such as hydroxypropylmethyl cellulose) or amino-alkylmethacrylate copolymers (e.g. Eudragit™ E). In some embodiments, the coating agent comprises Kollicoat® IR (a polyvinyl alcohol-polyethylene glycol graft copolymer) or Kollicoat IR White®, both manufactured by BASF Aktiengesellschaft (Ludwigshafen, Germany). In some embodiments, the coating agent comprises OPADRY II blue.

In some embodiments, the pharmaceutical composition is in the form of a tablet. In some embodiments, the pharmaceutical composition is in the form of a scored tablet.

In some embodiments, the pharmaceutical composition is substantially spherical in shape. In some embodiments, the pharmaceutical composition is substantially cylindrical in shape. In some embodiments, the pharmaceutical composition is substantially capsule-shaped.

The pharmaceutical compositions described herein are stable for extended periods under “forced degradation” conditions of elevated temperature and relative humidity. For example, pharmaceutical compositions of famotidine and ibuprofen prepared as described herein may exhibit improvements in stability at 40° C. and 75% relative humidity, relative to alternative designs (e.g., barrier-coated famotidine multiparticulates in a matrix comprising ibuprofen).

“Forced degradation” conditions (e.g., 40° C. and 75% relative humidity) may be used to evaluate the long-term storage stability of a pharmaceutical ingredient or composition. In general terms, a stable composition is one which comprises the pharmaceutically active ingredients in an amount, for example about 95%, relative to the amount initially present in the particular composition. Stability may be determined, using forced degradation or other methods, for periods of about 1 week, about 1 month, about 2 months, about 3 months, about 4 months, about 5 months, about 6 months, about 9 months, about 12 months, about 15 months, about 18 months, about 24 months, about 30 months, about 36 months, or for a longer period of time that will be apparent to a skilled artisan.

Stability may also be determined by the presence and quantity of impurities. A principal degradant produced through the chemical interaction of famotidine and ibuprofen in pharmaceutical compositions described herein is sulfamide. A quantitative determination of the presence of sulfamide in a pharmaceutical composition described herein held under forced degradation conditions for a period of time yields valuable information about the long-term stability of the composition under ordinary (e.g., room temperature) storage conditions. In some embodiments, the pharmaceutical composition retains ibuprofen and famotidine contents of at least about 90%, and such as at least about 95%, at least about 96%, at least about 97%, at least about 98%, at least about 99%, or at least about 99.5% of the originally specified amount for each such ingredient after about 1, about 3, about 6, about 9, or about 12 months at about 40° C. and about 75% relative humidity. In some embodiments, the pharmaceutical composition retains ibuprofen and famotidine contents of at least about 90%, and such as at least about 95%, at least about 96%, at least about 97%, at least about 98%, at least about 99%, or at least about 99.5% of the originally specified amount for each such ingredient after about 1, about 3, about 6, about 9, or about 12 months at about 25° C. and about 60% relative humidity. In further embodiments, after about 1, about 3, about 6, about 9, or about 12 months at either about 40° C. and about 75% relative humidity or about 25° C. and about 60% relative humidity, the pharmaceutical compositions contain no more than about 2%, about 1%, about 0.9%, about 0.8%, about 0.7%, about 0.6%, about 0.5%, about 0.4%, about 0.3%, about 0.2%, or about 0.1% sulfamide. In some circumstances, total impurities are present at no more than about 3%, about 2%, about 1%, about 0.5%, about 0.4%, about 0.3%, or about 0.2% after the time periods and conditions listed above.

Assays for evaluating the stability of a pharmaceutical composition, such as those described herein, are known in the pharmaceutical arts. For example, one can determine the percentage of active pharmaceutical ingredients present in a given composition, as well as the presence and percentage of impurities, through the use of standard analytical techniques.

The pharmaceutical compositions described herein are formulated so that release of both (ibuprofen and the H₂RA) active pharmaceutical ingredients (APIs) occurs (or begins to occur) at about the same time. “At about the same time” means that release of one API begins within 5 minutes of the beginning of release of the second API, sometimes with 4 minutes, sometimes within 3 minutes, sometimes within 2 minutes, and sometimes essentially simultaneously. “At about the same time” can also mean that release of one API begins before release of the second API is completed. That is, the dosage form is not designed so that one of the APIs is released significantly later than the other API. To achieve this, combinations of excipients (which may include one or more of a binder, a lubricant, a disintegrant, a glidant and other components) are selected that do not retard or substantially retard the release of an API.

In the pharmaceutical compositions described herein, both the H₂RA or ibuprofen are formulated for immediate release, and not for release profiles commonly referred to as delayed release, sustained release, or controlled release. In some embodiments, the unit dosage form is formulated so that H₂RA and ibuprofen are released rapidly under neutral pH conditions (e.g., an aqueous solution at about pH 6.8 to about pH 7.4, e.g., about pH 7.2). In this context, “rapidly” means that both APIs are significantly released into solution within about 20 minutes under in vitro assay conditions. In some embodiments both APIs are significantly released into solution within about 15 minutes under in vitro assay conditions. In this context, “significantly released” means that at least about 60% of the weight of the API in the unit dosage form is dissolved, or at least about 75%, or at least about 80%, or at least about 90%, and sometimes at least about 95%. In some embodiments, both H₂RA and ibuprofen are at least about 95% released in about 30 minutes.

Dissolution rates may be determined using known methods. Generally an in vitro dissolution assay is carried out by placing the H₂RA-ibuprofen unit dosage form(s) (e.g., tablet(s)) in a known volume of dissolution medium in a container with a suitable stirring device. Samples of the medium are withdrawn at various times and analyzed for dissolved active substance to determine the rate of dissolution. Dissolution may be measured, for example, as described for ibuprofen in the USP or, alternatively, as described for famotidine or another H₂RA in the USP. Briefly, in this exemplary method, the unit dose form (e.g., tablet) is placed in a vessel of a United States Pharmacopeia dissolution apparatus II (Paddles) containing 900 ml dissolution medium at 37° C. The paddle speed is 50 RPM. Independent measurements are made for at least three (3) tablets. In one suitable in vitro assay, dissolution is measured using a neutral dissolution medium such as 50 mM potassium phosphate buffer, pH 7.2 (“neutral conditions”).

Alternatively, dissolution rates may be determined under low pH conditions. Release under low pH conditions can be measured using the in vitro dissolution assay described above, but using, for example, 50 mM potassium phosphate buffer, pH 4.5, as a dissolution medium. As used in this context, the APIs are released rapidly at low pH when a substantial amount of both APIs is released into solution within 60 minutes under low pH assay conditions. In some embodiments, a substantial amount of both APIs is released into solution within 40 minutes under low pH assay conditions. In some embodiments, a substantial amount of both APIs is released into solution within 20 minutes under low pH assay conditions. In some embodiments, a substantial amount of both APIs is released into solution within 10 minutes under low pH assay conditions. In this context, a “substantial amount” means at least about 15%, or at least about 20%, or at least about 25% of ibuprofen is dissolved and at least about 80%, or at least about 85%, or at least about 90% of the H₂RA is dissolved.

The pharmaceutical compositions described herein are suitable for three times per day (TID) administration of the H₂RA and ibuprofen to a subject in need thereof. Other pharmaceutical compositions suitable for administration at other frequencies will be apparent to the skilled artisan upon reading this disclosure.

In some embodiments, the H₂RA is ranitidine. In some embodiments, the first compartment comprises from about 25 mg to about 100 mg of ranitidine. In some embodiments, the first compartment comprises about 25 mg, about 50 mg, about 75 mg, or about 100 mg ranitidine. In some embodiments, the first compartment comprises from about 12.5 mg to about 50 mg of ranitidine.

In some embodiments, the H₂RA is famotidine. In some embodiments, the first compartment comprises about 24 mg to about 28 mg of famotidine. In some embodiments, the first compartment comprises about 26.6 mg of famotidine.

In some embodiments, the first compartment comprises, about 13 mg to about 15 mg of famotidine. In some embodiments, the first compartment comprises about 6.5 mg, about 13 mg, about 19.5 mg, or about 26 mg of famotidine.

In some embodiments, the first compartment comprises about 70 mg, about 60 mg, about 50 mg, about 40 mg, or about 30 mg of one or more excipients. In some embodiments, the excipient comprises one or more of microcrystalline cellulose, at least one binder other than microcrystalline cellulose, and a lubricant. In some embodiments, the excipients are free of or substantially free of starch.

In some embodiments, the first compartment comprises about 44 mg of microcrystalline cellulose. In some embodiments, the microcrystalline cellulose may be replaced, completely or in part by another binder.

In some embodiments, the first compartment comprises about 10 mg to about 15 mg of at least one binder other than microcrystalline cellulose. In some embodiments, the first compartment comprises about 12 mg to about 14 mg of at least one binder other than microcrystalline cellulose. In some embodiments, the at least one binder other than microcrystalline cellulose is lactose. In some embodiments, the pharmaceutical composition is free of or substantially free of lactose.

In some embodiments, the first compartment comprises about 1.4 mg of at least one lubricant. In some embodiments, the lubricant is magnesium stearate.

In some embodiments, the first compartment contains from about 42 mg to about 46 mg of microcrystalline cellulose, from about 10 to about 15 mg of lactose (anhydrous), from about 4 to about 6 mg of croscarmellose sodium, from about 0.1 to about 1 mg colloidal silicon dioxide, and from about 0.9 mg to about 1.9 mg of magnesium stearate. In some embodiments, the first compartment contains about 44 mg of microcrystalline cellulose, about 13 mg of lactose (anhydrous), about 5 mg of croscarmellose sodium, about 0.5 mg colloidal silicon dioxide, and about 1.4 mg of magnesium stearate.

In some embodiments, the first compartment comprises one or more additional excipients.

In some embodiments, the first compartment comprises
from about 24 mg to about 28 mg famotidine;
from about 42 mg to about 46 mg of microcrystalline cellulose;
from about 10 mg to about 19 mg of at least one binder other than microcrystalline cellulose; and
from about 0.9 mg to about 1.9 mg of at least one lubricant.

In some embodiments, the first compartment comprises
from about 40 to about 45 parts w/w famotidine;
from about 85 to about 90 parts w/w microcrystalline cellulose, such as Avicel PH 102;
from about 20 to about 30 parts w/w at least one filler, such as lactose, for example lactose monohydrate; and
from about 2 to about 5 parts w/w lubricant.

In some embodiments, the first compartment comprises from about 6.9 wt % to about 7.9 wt % of the total weight of the pharmaceutical composition.

In some embodiments, the first compartment is coated to provide a coated first compartment. In some embodiments, the coated first compartment comprises from about 7.4 wt % to about 8.4 wt % of the total weight of the pharmaceutical composition. In some embodiments, the coated first compartment comprises from about 7.1 wt % to about 8.1 wt % of the total weight of the pharmaceutical composition.

In some embodiments, the coated first compartment has a surface area that does not exceed about 40 mm², about 30 mm², about 20 mm², or about 10 mm².

In some embodiments, the coated first compartment is substantially cylindrical in shape. In some embodiments, the coated first compartment is substantially cylindrical in shape, and the radius of the cylinder approximates the length. In some embodiments, the coated first compartment has a surface area that does not exceed about 120 mm², about 119 mm², about 118 mm², about 117 mm², about 116 mm², about 115 mm², about 114 mm², about 113 mm², about 112 mm², about 111 mm², or about 110 mm².

In some embodiments, the coated first compartment is substantially spherical in shape. In some embodiments, the coated first compartment has a surface area that does not exceed about 100 mm², about 99 mm², about 98 mm², about 97 mm², about 96 mm², about 95 mm², about 94 mm², about 93 mm², about 92 mm², about 91 mm², or about 90 mm².

In some embodiments, the coated first compartment is capsule-shaped or substantially capsule shaped. As an illustrative example, Ambien® (zolpidem tartarate) 5 and 10 mg unit dose forms are available in capsule shaped tablets.

In some embodiments, the second compartment comprises from about 775 mg to about 825 mg of ibuprofen. In some embodiments, the second compartment comprises about 800 mg of ibuprofen.

In some embodiments, the second compartment comprises about 100 mg, about 150 mg, about 200 mg, about 250 mg, about 300 mg, about 350 mg, about 400 mg, or about 450 mg ibuprofen.

In some embodiments, the ibuprofen and famotidine are in a ratio of from about 29:1 to about 31:1, and such as in a ratio of about 30:1.

In some embodiments, the ibuprofen is in the form of Ibuprofen DC85™. In some embodiments, the mean particle size of ibuprofen ranges from about 25 to about 50 microns. In some embodiments, the second compartment consists essentially of Ibuprofen DC 85™. In some embodiments, the second compartment comprises
from about 99% to about 99.8% Ibuprofen DC85™ and
from about 0.2% to about 1% of at least one lubricant, such as magnesium stearate. In some embodiments, the second compartment comprises
from about 99.5% to about 99.8% Ibuprofen DC 85™ and
from about 0.2% to about 0.5% of at least one lubricant, such as magnesium stearate. In some embodiments, about 0.25% of at least one lubricant is present in the second compartment.

In some embodiments, the second compartment comprises
from about 750 mg to about 850 mg Ibuprofen DC 85™;
from about 160 to about 180 mg of at least one binder; and
from about 2.5 mg to about 3.5 mg of at least one lubricant.

In some embodiments, the second compartment contains from about 750 mg to about 850 mg ibuprofen, from about 160 to 180 mg of microcrystalline cellulose, from about 50 to about 60 mg of povidone, and from about 2.5 mg to about 3.5 mg of magnesium stearate. In some embodiments, the second compartment contains about 941 mg Ibuprofen DC 85™, about 110 mg microcrystalline cellulose in addition to the microcrystalline cellulose present in Ibuprofen DC 85™, about 55 mg povidone, and about 2.8 mg magnesium stearate.

In some embodiments, the second compartment contains from about 94% to about 96% Ibuprofen DC 85™, from about 3.5% to about 5.5% of povidone, and from about 0.2% to about 0.5% magnesium stearate. In some embodiments, the second compartment contains about 94.75% Ibuprofen DC 85™, about 5% povidone, and about 0.25% magnesium stearate.

In some embodiments, the second compartment comprises from about 92.5% to about 95% Ibuprofen DC 85™, about 4.5% to about 6.5% microcrystalline cellulose, such as Avicel PH 102, and from about 0.2% to about 0.5% of at least one lubricant, such as magnesium stearate. In some embodiments, the second compartment comprises about 93.75% Ibuprofen DC 85™, about 6% microcrystalline cellulose, such as Avicel PH 102, and about 0.25% of at least one lubricant, such as magnesium stearate.

In some embodiments, the second compartment comprises from about 88% to about 92% Ibuprofen DC 85™, about 8% to about 11% microcrystalline cellulose, such as Avicel PH 101 or Avicel PH 200, and from about 0.2% to about 0.5% of at least one lubricant, such as magnesium stearate. In some embodiments, the second compartment comprises about 89.75% Ibuprofen DC 85™, about 10% microcrystalline cellulose, such as Avicel PH 101 or Avicel PH 200, and about 0.25% of at least one lubricant, such as magnesium stearate.

In some embodiments, the second compartment comprises about 167 mg of at least one binder. In some embodiments, the at least one binder is chosen from microcrystalline cellulose and povidone. In some embodiments, the at least one binder is one or more of Avicel PH 102, Avicel PH 200, Avicel PH 101, and Avicel PH 105 microcrystalline cellulose, Klucel EXF hydroxypropyl cellulose, propylene glycol, Starch 1500, Lubritab, Kollidon VA 64 vinylpyrrolidone-vinyl acetate copolymer, PVP K 30 polyvinyl pyrrolidone, sodium stearyl fumarate, and stearic acid. In some embodiments, the binder is one or more of Avicel PH 105 microcrystalline cellulose, hydroxypropyl cellulose, or propylene glycol. In some embodiments, the binder is Avicel PH 101 or PH 105 microcrystalline cellulose, and one or more of Kollidon VA 64 vinylpyrrolidone-vinyl acetate copolymer, PVP K 30 polyvinyl pyrrolidone, or Klucel EXF hydroxypropyl cellulose.

In some embodiments, the second compartment comprises one or more additional excipients. Pharmaceutically acceptable excipients useful in pharmaceutical compositions described herein can include binders, lubricants, disintegrants, and glidants, or the like, as known in the art.

In some embodiments, the second compartment comprises about 2.8 mg of at least one lubricant. In some embodiments, the at least one lubricant comprises magnesium stearate.

In some embodiments, the second compartment comprises 941.2 mg Ibuprofen DC 85™ (which provides about 800 mg ibuprofen) and one or more of the following excipients: magnesium stearate, Avicel PH 105, Klucel EXF, propylene glycol, starch 1500, lubritab, sodium stearyl fumarate, and stearic acid. In some embodiments, the second compartment comprises 941.2 mg Ibuprofen DC 85™ (which provides about 800 mg ibuprofen) and about 22.4 mg magnesium stearate. In some embodiments, the second compartment comprises 941.2 mg Ibuprofen DC 85™ (which provides about 800 mg ibuprofen) and about 104.6 mg Avicel PH 105. In some embodiments, the second compartment comprises 941.2 mg Ibuprofen DC 85™ (which provides about 800 mg ibuprofen) and about 83.7 mg Avicel PH 105. In some embodiments, the second compartment comprises 941.2 mg Ibuprofen DC 85™ (which provides about 800 mg ibuprofen) and about 104.6 mg Klucel EXF. In some embodiments, the second compartment comprises 941.2 mg Ibuprofen DC 85™ (which provides about 800 mg ibuprofen) and about 20.9 mg propylene glycol. In some embodiments, the second compartment comprises 941.2 mg Ibuprofen DC 85™ (which provides about 800 mg ibuprofen) and about 104.6 mg starch 1500. In some embodiments, the second compartment comprises 941.2 mg Ibuprofen DC 85™ (which provides about 800 mg ibuprofen) and about 29.1 mg lubritab. In some embodiments, the second compartment comprises 941.2 mg Ibuprofen DC 85™ (which provides about 800 mg ibuprofen) and about 19.2 mg sodium stearyl fumarate. In some embodiments, the second compartment comprises 941.2 mg Ibuprofen DC 85™ (which provides about 800 mg ibuprofen) and about 19.2 mg stearic acid.

In some embodiments, the second compartment comprises about 84.75% Ibuprofen DC 85™, 7.00% Avicel PH 101, 8.00% Kollidone VA-64, and 0.25% magnesium stearate, each on a w/w basis. In some embodiments, the second compartment comprises about 81.75% Ibuprofen DC 85™, 10.00% Avicel PH 105, 8.00% PVP K30, and 0.25% magnesium stearate, each on a w/w basis. In some embodiments, the second compartment comprises about 81.75% Ibuprofen DC 85™, 10.00% Avicel PH 105, 8.00% Kollidone VA-64, and 0.25% magnesium stearate, each on a w/w basis. In some embodiments, the second compartment comprises about 84.75% Ibuprofen DC 85™, 5.00% Avicel PH 105, 10% PVP K30, and 0.25% magnesium stearate, each on a w/w basis. In some embodiments, the second compartment comprises about 83.75% Ibuprofen DC 85™, 10.00% Avicel PH 105, 6.00% Klucel EXF and 0.25% magnesium stearate, each on a w/w basis.

In some embodiments, the second compartment comprises from about 91.6 wt % to about 92.6 wt % of the total weight of the pharmaceutical composition.

In some embodiments, a barrier layer is used to separate the first compartment from the second compartment. In some embodiments, the barrier layer comprises a water-soluble, substantially pH independent film that promotes immediate disintegration for rapid release of the coated drug (i.e., ibuprofen and/or the H₂RA). In some embodiments, the barrier layer comprises a film readily soluble in an aqueous media. Materials that can be used for readily soluble films are well known in the art and include cellulose derivatives such as hydroxypropylmethyl cellulose, hydroxypropyl cellulose, hydroxypropyl methylcellulose phthalate, cellulose acetate phthalate, and ethyl cellulose, and vinyl polymers such as methacrylic polymers, amino-alkylmethacrylate copolymers (e.g. Eudragit™ E), polyvinyl acetate phthalate and polyvinyl alcohol (PVA), and combinations of each thereof.

In some embodiments, a plasticizer (e.g., triacetin, diethyl phthalate, tributyl sebacate or polyethylene glycol) is included.

In some embodiments, the barrier layer may include an anti-adherent or glidant (e.g., talc, fumed silica or magnesium stearate) and colorants such as titanium dioxide, iron oxide based colorants or others. In some embodiments, the barrier layer is colored.

In some embodiments the barrier layer further comprises a non-toxic edible polymer, edible pigment particles, an edible polymer plasticizer, or a surfactant.

In some embodiments, the barrier layer comprises materials described, for example, U.S. Pat. No. 4,543,370 (Colorcon). Exemplary barrier layers include OPADRY®, OPADRY White (Product number YS-1-7003), and OPADRY II® which are available from Colorcon (West Point Pa. USA); and polyvinyl alcohol-polyethylene glycol copolymer marketed as Kollicoat® IR (BASF). Suitable barrier layers, for illustration and not limitation, include Kollicoat® IR (a polyvinyl alcohol-polyethylene glycol graft copolymer) and Kollicoat IR White® both manufactured by BASF Aktiengesellschaft (Ludwigshafen, Germany).

In some embodiments, the barrier layer comprises Opadry White. In some embodiments, the barrier layer comprises Opadry White and Opadry White II. In some embodiments, the barrier layer comprises Kollicoat. In some embodiments, the barrier layer comprises Eudragit™ E (polyacrylates), Klucell® (hydroxypropyl cellulose), or Povidone® (polyvinyl pyrrolidone). In some embodiments, the barrier layers comprises a wax.

The thickness of the barrier layer can vary over a wide range, but in some embodiments, is in the range of from 20 to 3,000 microns, such as on the order of about 25 to 250 microns.

In some embodiments, the barrier layer is about 2% to about 25%, about 2% to about 15%, about 2% to about 8%, or about 2% to about 5.5% based on the weight of the core. In some embodiments, the core tablet is coated with the barrier layer for a weight gain of about 5% to about 6%. In some embodiments, the core tablet is coated with the barrier layer for a weight gain of about 5.5%.

In some embodiments, the barrier layer retards the release of API by less than about 5 minutes, such as less than about 4 minutes, for example, by less than about 3 minutes.

In some embodiments, the barrier layer is soluble in an aqueous media, such as, for example, water. In some embodiments, the barrier layer is soluble in organic solvent, such as ethanol, methanol, and the like.

In some embodiments, none of the pharmaceutical composition, the famotidine, and the ibuprofen is enterically coated or formulated for sustained or delayed release.

Also provided is a process for preparing a pharmaceutical composition comprising an ibuprofen shell completely surrounding a coated core tablet wherein the coated core tablet comprises an H₂RA and a barrier layer, wherein said process comprises

blending a therapeutically effective amount of an H₂RA with at least one pharmaceutically acceptable excipient to yield a blended H₂RA mixture;

pressing said blended H₂RA mixture;

coating said pressed blended H₂RA mixture with a barrier layer to yield a coated core tablet;

blending a therapeutically effective amount of ibuprofen with at least one pharmaceutically acceptable excipient to yield a blended ibuprofen mixture;

optionally granulating said blended ibuprofen mixture to yield a granulated ibuprofen; and

compressing said blended ibuprofen mixture or said granulated ibuprofen around the coated core tablet to yield an ibuprofen shell such that the ibuprofen shell completely surrounds the coated core tablet.

In some embodiments, the process further comprises coating said ibuprofen shell with a coating agent to yield a coated pharmaceutical composition.

In some embodiments, it is contemplated that the coated core tablet comprising an H₂RA is prepared by blending a therapeutically effective amount of the H₂RA with at least one pharmaceutically acceptable excipient to yield a blended H₂RA mixture, spray coating said blended H₂RA mixture on a core to yield a H₂RA coated core, and coating said H₂RA coated core with a barrier layer to yield a coated core tablet. In some embodiments, the core comprises a sugar, such as sucrose, lactose, and the like. The coated core thus prepared can be converted to a core-shell or a tablet-in-tablet pharmaceutical composition provided here by adding an ibuprofen shell as provided here.

In one embodiment, the granulating is performed by dry granulation. In one embodiment, the granulating is performed by wet granulation. When employing wet granulation, ibuprofen of any mean particle size may be advantageously employed to manufacture the compositions provided herein.

In some embodiments, the bilayer or trilayer tablets are manufactured using a tablet press, and charging one of the hoppers with a blend comprising ibuprofen that is provided herein and another hopper with a blend comprising an H₂RA that is provided herein and running the press for suitable periods of time, which time periods are well known to the skilled artisan or will be apparent to the skilled artisan upon reading this disclosure.

Also provided is a pharmaceutical composition prepared by any of the processes described herein.

Also provided are methods of treating subjects in need of ibuprofen and an H₂RA treatment. Ibuprofen is indicated for treatment of mild to moderate pain, dysmenorrhea, inflammation, and arthritis, including with limitation relief of the signs and symptoms of rheumatoid arthritis and osteoarthritis.

In some embodiments, the subject in need of ibuprofen treatment is under treatment for a chronic condition. For example and without limitation, a subject in need of ibuprofen treatment may be a subject with rheumatoid arthritis, a subject with osteoarthritis, a subject suffering from chronic pain (e.g., chronic low back pain, chronic regional pain syndrome, chronic soft tissue pain), or a subject suffering from a chronic inflammatory condition. In general, a subject under treatment for a chronic condition requires ibuprofen treatment for an extended period, such as at least one month, at least four months, at least six months, or at least one year, and at least some of these subjects can benefit from receiving famotidine in combination with ibuprofen during such treatment period.

In some embodiments, the subject in need of ibuprofen treatment is under treatment for a condition that is not chronic, such as acute pain, dysmenorrhea or acute inflammation.

In some embodiments, the subject has a Body Mass Index in the normal range.

In some embodiments, the subject is at elevated risk of developing an NSAID-induced ulcer (i.e., the subject is more susceptible than the average individual to development of an ulcer when under treatment with an NSAID).

In some embodiments, the subjects are pediatric subjects. When used for treating such subjects, the amount of famotidine (or another H₂RA) and ibuprofen employed are typically in the lower end of the dose ranges disclosed here. For example, and without limitation, pharmaceutical compositions containing about 13 mg to about 15 mg of famotidine and about 100 mg to about 450 mg of ibuprofen are useful fort treating such subjects. To provide a pharmaceutical composition that is smaller and/or easier to administer to pediatric subjects, such compositions are contemplated to include smaller amounts of excipients, and easily swallowable compositions comprising, e.g., soft gel.

In some embodiments, the subject in need of H₂RA treatment is at risk of developing upper gastrointestinal ulcers, which include gastric and/or duodenal ulcers. In some embodiments, the subject in need of H₂RA treatment requires treatment for non-ulcerative dyspepsia. In some embodiments, the subject in need of H₂RA treatment requires treatment for gastroesophageal reflux disease (GERD) or for esophagitis due to GERD or for ulcer (duodenal or gastric). In some embodiments, the subject in need of H₂RA treatment requires treatment for dyspepsia but does not require treatment for ulcer, GERD or its complications.

In some embodiments, the subject is not under NSAID therapy (e.g., does not take ibuprofen and/or a different NSAID for treatment of a chronic condition). In some embodiments, the subject in need of ibuprofen treatment does not suffer from a condition characterized by hypersecretion of gastric acid (e.g., Zollinger-Ellison Syndrome). In some embodiments, the subject does not suffer from Barrett's ulceration or active severe oesophagitis.

In some embodiments, the subject does not have gastroesophageal reflux disease (GERD) or esophagitis due to GERD. In some embodiments, the subject is not in need of treatment for an ulcer. In some embodiments, the subject does not suffer from dyspepsia.

Provided is a method for administration of ibuprofen to a subject in need of ibuprofen treatment comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method for reducing the incidence of NSAID-induced gastric and/or duodenal ulcers comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method for reducing gastric acid while treating a subject with an ibuprofen-responsive condition comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method for treating a subject in need of ibuprofen treatment, where the subject is at elevated risk for developing an NSAID-induced ulcer comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method for reducing symptoms of dyspepsia in a subject in need of NSAID treatment who has experienced symptoms of dyspepsia associated with NSAID administration comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method of reducing or preventing the occurrence of gastrointestinal toxicity associated with the use of ibuprofen, such as gastrointestinal ulceration and dyspepsia comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method of treating a subject in need of ibuprofen treatment, where the subject is at high risk for developing an NSAID-induced ulcer comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method of reducing, in a subject in need of ibuprofen treatment, the risk of developing an ibuprofen-induced symptom or condition such as ulcer or GERD comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method of reducing symptoms of a famotidine-responsive condition, such as dyspepsia, in a subject in need of NSAID treatment who has experienced symptoms of a famotidine-responsive condition, such as dyspepsia, associated with NSAID administration, comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

Also provided is a method for preventing toxicities associated with ibuprofen use in subjects who are specifically at risk for the development of such toxicities comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

In some embodiments, the method further comprises, prior to administering the pharmaceutical composition, determining an approximate serum creatinine concentration for the individual; if the subject has a creatinine clearance rate of greater than about 50 mL/minute, then prescribing or administering a first dose of the pharmaceutical composition described herein.

In some embodiments, the method further comprises, prior to prescribing or administering the pharmaceutical composition, determining if the subject is being administered one or more additional therapeutic agents chosen from diuretics, angiotensin converting enzyme inhibitors, and angiotensin receptor blockers, if the subject is being administered one or more of said additional therapeutic agents, then determining an approximate creatinine clearance rate for the individual; then if the subject has a creatinine clearance rate of greater than about 50 mL/minute, prescribing or administering a first dose of a pharmaceutical composition described herein.

Also provided is a method for reducing the risk of an adverse event in an subject requiring ibuprofen for an ibuprofen-responsive comprising:
a) determining an approximate serum creatinine concentration for the individual;
b) if the subject has a creatinine clearance rate of greater than about 50 mL/minute, then prescribing or administering a first dose of a pharmaceutical composition described herein;
c) prescribing or administering to the human subject a second dose of the pharmaceutical composition; and
d) prescribing or administering to the human subject a third dose of the pharmaceutical composition.

Also provided is a method for the treatment of cystic fibrosis comprising prescribing or administering the pharmaceutical compositions described herein. In some embodiments, the subject is instructed to ingest the pharmaceutical composition three times daily. In some embodiments, the subject is instructed to ensure there is at least a 6-hr interval between administrations of consecutive doses.

In some embodiments, the method further comprises administering one or more of therapeutic agents chosen from an antibiotic, anti-inflammatory, bronchodilatory, antihistamine or anti-tussive drug substances to the subject. In some embodiments, the antibiotic substance is chosen from astreonam, ceftazidime, tobramycin, and ciprofloxacin. In some embodiments, the one or more the one or more therapeutic agents is administered by inhalation.

Thus, also provided is a method in which TID administration of a dosage form containing about 800 mg ibuprofen and about 26.6 mg famotidine provides better gastric protection over a 24-hour period than TID administration of the 800 mg ibuprofen and BID administration of 40 mg famotidine. Equivalently, TID administration of two oral dosage forms containing about 400 mg ibuprofen and about 13 mg (e.g., about 13.3 mg) famotidine provides better gastric protection over a 24-hour period than TID administration 800 mg ibuprofen in a single or split dose and BID administration of 40 mg famotidine in a single or split dose.

In some embodiments, the subject is not also being administered a warfarin-type anticoagulants. In some embodiments, the subject is not also being administered an NSAID other ibuprofen. In some embodiments, the subject is not also being administered aspirin. In some embodiments, the subject is not also being administered an ACE-inhibitor or diuretic such as thiazides or loop diuretics. In some embodiments, the subject is not also being administered lithium. In some embodiments, the subject is not also being administered methotrexate. In some embodiments, the subject is not also being administered a selective serotonin reuptake inhibitor. In some embodiments, the subject is not also being administered cholestyramine.

In some embodiments, the subject does not have cardiovascular disease or a risk factor for cardiovascular disease. In some embodiments, the subject is less than 65 years of age and/or without a prior history of gastrointestinal ulcer.

In some embodiments, the subject does not have hypertension.

In some embodiments, the subject does not have fluid retention or heart failure.

In some embodiments, the subject is also being administered oral corticosteroids. In some embodiments, the subject is also being administered anticoagulants. In some embodiments, the subject is also being administered antiplatelet drugs (including low-dose aspirin). In some embodiments, the subject smokes. In some embodiments, the subject uses alcohol. In some embodiments, the subject is in poor general health.

In some embodiments, the subject has a history of inflammatory bowel disease including ulcerative colitis and Crohn's disease.

In some embodiments, the method further comprises monitoring blood pressure of the subject.

In some embodiments, the method further comprises monitoring for signs or symptoms of gastrointestinal bleeding. In some embodiments, the method further comprises assessing the subject's complete blood count and chemistry profile periodically.

In some embodiments, the method further comprises determining the subject's hemoglobin value. In some embodiments, if the subject has an initial hemoglobin value of 10 g or less, the method further comprises determining the subject's hemoglobin value periodically.

In some embodiments, the method further comprises determining whether the subject exhibits clinical signs and symptoms consistent with renal disease, such as azotemia, hypertension and/or proteinuria.

In some embodiments, the method further comprises determining whether the subject exhibits any appearance of skin rash or any other sign of hypersensitivity.

In some embodiments, the method further comprises determining the subject's level of alanine aminotransferase (ALT) or aspartate aminotransferase (AST).

### EXAMPLES

The following examples are offered to illustrate, but not to limit, the claimed invention.

### I. Manufacture of Ibuprofen/Famotidine Unit Dose Forms

### (1) Formulation (A)

This example describes manufacture of a tablet containing ibuprofen granules and coated famotidine granules.

a. Ibuprofen Granules

TABLE 1

| | | | | % of |
|---|---|---|---|---|
| | | | Amount per | Tablet |
| Item | Component | Function | Tablet (mg) | (w/w) |
| 1 | Ibuprofen, USP | Active | 800.0 | 58.49 |
| 2 | Lactose | Binder | 300.0 | 21.93 |
| | monohydrate, NF | | | |
| 3 | Colloidal silicon | Glidant | 6.0 | 0.44 |
| | dioxide, NF | | | |
| 4 | Croscarmellose | Disintegrant | 30.0 | 2.19 |
| | sodium, NF | | | |
| 5 | Hypromellose, USP | Filler | 24.0 | 1.75 |
| 6 | Purified Water, USP | Solvent | * | * |

| | | |
|---|---|---|
| Total weight | 1160.0 | 84.80 |

* Water is removed during the process and therefore not factored in tablet weight.

Items 1-5 are sifted through Quadro Comil 16-mesh and mixed (Blend 1). Item 5B is dissolved in water and slowly added to Blend 1 using a mixer. Additional water is added and mixed. The wet material is dried at 50° C. for 12 h, milled using a 16-mesh screen with appropriate spacer, and dried until the LOD at 50° C. is below 0.5% w/w. Dried granules and extra granular material is transferred to a V-blender and mixed for 3 minutes.

b. Famotidine Granules

TABLE 2

| | | | | |
|---|---|---|---|---|
| | | | | % of |
| | | | Amount per | Tablet |
| Item | Component | Function | Tablet (mg) | (w/w) |

Famotidine Granules

| | | | | |
|---|---|---|---|---|
| 1 | Famotidine, USP | Active | 26.6 | 1.94 |
| 2 | Opadry II White | Coating | 7.1 | 0.52 |
| | (Y-22-7719) | agent | | |
| 3 | Talc, Imperial, USP | Thickening | 1.8 | 0.13 |
| | | agent | | |
| 4 | Microcrystalline | Binder | 35.5 | 2.60 |
| | cellulose, NF | | | |
| 5 | Purified Water, USP ** | Solvent | * | * |

| | | |
|---|---|---|
| Total weight | 71.0 | 5.19 |

Barrier Coating

| | | | | |
|---|---|---|---|---|
| 6 | Opadry White | Coating | 7.1 | 0.52 |
| | (YS-1-7003) | agent | | |
| 7 | Purified Water, USP *** | Solvent | * | * |

| | | |
|---|---|---|
| Total weight | 7.1 | 0.52 |

* Water is removed during the process and therefore not factored in tablet weight.
** Famotidine suspension is 20% solids w/w.
*** Protective coating suspension is 13% solids w/w.

Set up the Glatt fluid bed processor and add microcrystalline cellulose to Glatt. Disperse famotidine in purified water under mechanical stirring for 5 minutes. Add Opadry followed by talc and let it run for 30 minutes. Homogenize the above suspension for 20-30 minutes. Keep mixing at slow speed to avoid air entrapment.

Set up the peristaltic pump and spray the drug suspension completely. Dry the product to a product temperature of around 40-44° C. Sift the spray granulated famotidine through Quadro comil #20 mesh.

Spray Opadry suspension equivalent to 10% weight gain in the Glatt fluid bed processor. Dry the final product to a product temperature of around 40-44° C. Discharge and sift it through ASTM #30 mesh to remove any agglomerate.

c. Final Blending

TABLE 3

| | | | | % of |
|---|---|---|---|---|
| | | | Amount per | Tablet |
| Item | Component | Function | Tablet (mg) | (w/w) |
| 1 | Ibuprofen granules | In-process | 1160.0 | |
| | | granules | | |
| 2 | Famotidine granules | In-process | 78.1 | |
| | | granules | | |
| 3 | Colloidal silicon | Glidant | 4.0 | 0.29 |
| | dioxide, NF | | | |
| 4 | Croscarmellose | Disintegrant | 30.0 | 2.19 |
| | sodium, NF | | | |
| 5 | Silicified | Binder | 47.0 | 3.44 |
| | microcrystalline | | | |
| | cellulose (Prosolv | | | |
| | SMCC 90) | | | |
| 6 | Magnesium stearate, NF | Lubricant | 9.0 | 0.66 |

| | | |
|---|---|---|
| Total Tablet Weight | 1328.1 | 6.58 |

Weight appropriate amount of ibuprofen granules, famotidine granules and the extra-granular materials. Blend geometrically famotidine and ibuprofen granules in appropriate blenders.

Add the sifted extra-granular materials (Prosolv SMCC 90, croscarmellose sodium and colloidal silicon dioxide sifted through 16-mesh screen) to above granules and mix for 3 minutes.

Sift magnesium stearate through 30 mesh screen and transfer to the above blender and lubricate for 3 minutes.

d. Tabletting

Set DC-16 compression machine with bisect punches and compress the blend to tablets with target weight of 1.328 g, hardness of 10-20 Kp, disintegration time less than 15 minutes.

e. Film Coating

TABLE 4

| | | | | % of |
|---|---|---|---|---|
| | | | Amount per | Tablet |
| Item | Component | Function | Tablet (mg) | (w/w) |
| 1 | Ibuprofen/Famotidine | In-process | 1328.1 | |
| | core tablets | granules | | |
| 2 | Opadry II White | Film Coating | 39.9 | 2.91 |
| | (85F18422) | | | |
| 2 | Purified Water, USP | Solvent | * | * |

| | | |
|---|---|---|
| Total weight | 1368.0 | 100% |

* Water is removed during the process and therefore not factored in tablet weight.

Disperse Opadry II white (85 F18422) in water under mechanical stirring. Continue mixing for 45 minutes at slow speed. Load approximately 80-90 kg of compressed tablets in Acella Cota with a 48″ coating pan. Coat the tablets to a weight gain of 2.5-3.5% w/w following optimum coating parameters.

In other related embodiments tablets are made as above except that the amount of any non-API component can vary from the amounts above by up to plus or minus 10%. For example, the lactose monohydrate component in Table 1 could vary in the range from about 23.3 to about 28.4%. APIs can vary in amounts as described elsewhere herein.

f. Stability of Ibuprofen/Famotidine Tablet

Tablets were prepared as described above. The stability profile of the tablet is provided in Table 5.

TABLE 5

| | | | Ibuprofen | | Famotidine | | Total |
|---|---|---|---|---|---|---|---|
| | Time | | As- | 4- | As- | Sulf- | Impuri- |
| Conditions | Point | | say | IBAP¹ | say | amide | ties |
| 50° C. | 1 | week | 98.4 | 0.0 | 67.8 | 21.7 | 32.0 |
| 40° C./75% | 2 | weeks | 98.6 | 0.06 | 103.1 | 0.87 | 2.0 |
| RH | | | | | | | |
| | 1 | month | 98.6 | 0.0 | 99.9 | 3.2 | 4.4 |
| 25° C./60% | 1 | month | 99.6 | 0.0 | 105.1 | 0.1 | 0.3 |
| RH | | | | | | | |

### (2) Formulation (B): Tablet-In-Tablet

This example describes manufacture of a tablet containing ibuprofen granules and coated famotidine cores.

A tablet-in-tablet composition of famotidine and ibuprofen described herein can be prepared by first preparing a famotidine core, which is then coated with a barrier layer, then surrounded by an ibuprofen shell and an optional over-coating. The famotidine core is prepared by (i) combining 26.6 mg famotidine, 12.7 mg lactose, anhydrous, 44.1 mg microcrystalline cellulose, 4.7 mg croscarmellose sodium, and 0.7 mg colloidal silicon dioxide in a suitably sized V-blender; (ii) mixing the combined ingredients for approximately ten minutes; (iii) discharging the blended materials from the blender and passing them through a #20 mesh screen; (iv) transferring the screened material back into the V-blender and mixing for approximately ten additional minutes; (vi) passing 1.4 mg magnesium stearate through a #30 mesh screen; (vii) adding the screened magnesium stearate to the blended material in the V-blender and mixing for approximately three additional minutes; (viii) discharging the blended material into a polyethylene lined container; and (ix) compressing the blended material into a tablet (i.e., a famotidine core) on a rotary tablet press using 0.2187″ plain round SC tooling.

The famotidine tablet core is coated with a barrier layer by placement in a suitably sized perforated coating pan to which a dispersion of Opadry (YS-1-7003) (Colorcon) in water is added to coat the tablet core to a weight gain of 5.5%, which results in about 5 mg of added solids after drying.

After the barrier layer has dried, 941.2 mg of Ibuprofen DC 85 (containing 800 mg of ibuprofen) is blended with 111.1 mg microcrystalline cellulose, 55.5 mg povidone, and 2.8 mg magnesium stearate, then granulated. The granulated ibuprofen mixture is then compressed around the barrier-coated famotidine core using a tablet press. Finally, the tablet is over-coated by placement in a suitably sized perforated coating pan containing a dispersion of Opadry II blue (85F99093) (Colorcon) in water to coat the tablet to a weight gain of 4.0% (48.2 mg added solids after drying).

The composition of the tablet-in-tablet formulation (B) is provided in table 6.

TABLE 6

| | | | | |
|---|---|---|---|---|
| | | | | % of |
| | | | Amount per | Tablet * |
| Item | Component | Function | Tablet (mg) | (w/w) |

Famotidine Core Tablet

| | | | | |
|---|---|---|---|---|
| 1 | Famotidine, USP | Active | 26.6 | 2.1 |
| 2 | Microcrystalline | Binder | 44.1 | 3.5 |
| | cellulose, NF | | | |
| | (Avicel PH102) | | | |
| 3 | Lactose, anhydrous | Binder | 12.7 | 1.0 |
| | (SuperTab 21AN), NF | | | |
| 4 | Croscarmellose | Disintegrant | 4.7 | 0.4 |
| | sodium, NF | | | |
| | (Ac-Di-Sol SD-711) | | | |
| 5 | Colloidal silicon | Glidant | 0.5 | 0.04 |
| | dioxide, NF | | | |
| | (Cab-O-Sil M5P) | | | |
| 6 | Magnesium | Lubricant | 1.4 | 0.1 |
| | stearate, NF | | | |

| | | |
|---|---|---|
| Total weight | 90.0 | 7.2 |

Famotidine Core Tablet Coating

| | | | |
|---|---|---|---|
| Opadray white | Coating agent | 5.0 ** | 0.4 |
| (YS-1-7003) | | | |
| Purified | Solvent | *** | *** |
| water, USP | | | |

| | | |
|---|---|---|
| Total weight | 95.0 | 7.6 |

Ibuprofen Outer Tablet

| | | | | |
|---|---|---|---|---|
| 7 | Ibuprofen DC 85 | Active granules | 941.2 | 75.1 |
| 8 | Microcrystalline | Binder | 111.1 | 8.9 |
| | cellulose, NF | | | |
| | (Avicel PH101) | | | |
| 9 | Povidone, USP | Binder | 55.5 | 4.4 |
| | (Kollidon 30) | | | |
| 10 | Magnesium | Lubricant | 2.8 | 0.2 |
| | stearate, NF | | | |

| | | |
|---|---|---|
| Total weight | 1110.6 | 88.6 |

Table-in-Tablet Drug Product

| | | |
|---|---|---|
| Total weight | 1205.6 | 96.2 |

Tablet-in-Tablet Drug Product Coating

| | | | | |
|---|---|---|---|---|
| 6 | Opadry II blue | Coating agent | 48.2 **** | 3.8 |
| | (85F99093) | | | |
| 7 | Purified | Solvent | *** | *** |
| | Water, USP *** | | | |

| | | |
|---|---|---|
| Total coated tablet-in tablet weight | 1253.8 | 100% |

NF = National Formulary; USP = United States Pharmacopoeia
* Percent of the total weight.
** Tablets are coated to a weight gain target of 5.5% of total; amount represents solids remaining after drying.
*** Solvent is removed during the processing.
**** The finished tablets are coated to a weight gain of 4.0%. The amount represents the solids left after drying.

Stability of a tablet-in-tablet composition containing a famotidine core, Opadry white barrier layer, ibuprofen shell, and Opadry II blue overcoat was tested at 25° C./60% RH for 0, 3, 6, and 9 months. The tablets contained the same active ingredients as the tablets described in Example 1. The stability results of three batches of tablets based on the total % impurities present at each time point are shown below.

Total Impurities Stability Profile of the Tablet-in-Tablet Formulation
90-ct at 25 ± 2° C./60 ± 5% RH

Stability Interval (Months)

| | | | |
|---|---|---|---|
| Initial | 3 | 6 | 9 |

| | |
|---|---|
| Lot | Total Impurities (%) |

| | | | | |
|---|---|---|---|---|
| 001B | 0.2 | 0.1 | 0.2 | 0.2 |
| 004B | 0.1 | 0.1 | 0.2 | 0.3 |
| 005B | 0.1 | 0.1 | 0.2 | 0.2 |

In addition, the tablet-in-tablet plus barrier layer formulation was compared to a formulation without the barrier layer, as well as a formulation containing coated famotidine granules compressed within ibuprofen. Both sulfamide and total impurities were measured after 1 week at 50° C. and 1 month at 40° C. Under the test conditions, the tablet-in-tablet with barrier layer showed lowered levels of sulfamide formation as shown below.

Stability Evaluation of Tablet-in-Tablet Prototypes

| | | Famotidine | | | | |
|---|---|---|---|---|---|---|
| | Condi- | | Sulf- | Ibuprofen | | |
| | tion/ | | amide | | Total | Total |
| Lot | Time | Assay | Impuri- | Assay | Impuri- | Impuri- |
| No | Point | (%) | ty (%) | (%) | ties (%) | ties (%) |

Tablet-in-tablet (DC85 + DC Famotidine w/barrier layer)

| | | | | | | |
|---|---|---|---|---|---|---|
| 014 | Initial | 101.0 | 0.0 | 98.6 | 0.2 | 0.2 |
| | 1 wk 50° C. | 98.4 | 0.19 | 98.8 | 0.1 | 0.3 |
| | 1 mo 40° C. | 96.7 | 0.0 | 100.8 | 0.0 | 0.0 |

Tablet-in-tablet (DC85 + DC Famotidine w/o barrier layer)

| | | | | | | |
|---|---|---|---|---|---|---|
| 015 | Initial | 98.1 | 0.0 | 99.1 | 0.1 | 0.1 |
| | 1 wk 50° C. | 95.4 | 0.74 | 98.3 | 0.1 | 0.9 |
| | 1 mo 40° C. | 94.6 | 0.56 | 99.5 | 0.1 | 0.7 |

Bilayer (DC85 + coated famotidine granules)

| | | | | | | |
|---|---|---|---|---|---|---|
| 016 | Initial | 105.3 | 0.0 | 99.2 | 0.0 | 0.0 |
| | 1 wk 50° C. | 106.7 | 1.28 | 98.9 | 0.1 | 1.4 |
| | 1 mo 40° C. | 103.2 | 0.91 | 100.5 | 2.0 | 3.0 |

### II. Administration of Ibuprofen/Famotidine Tablets Reduces the Risk of NSAID-Associated Gastric and Duodenal Ulcers

### A. Methods

Subjects 40-80 yrs, expected to require daily NSAID therapy for a period of longer than six months for conditions such as osteoarthritis, rheumatoid arthritis, chronic low back pain, chronic regional pain syndrome, and chronic soft tissue pain; had no history of ulcer complications; had negative H. pylori stool test; and had baseline endoscopy showing no ulcers and less than five erosions in the upper GI tract were randomly assigned in a 2:1 ratio to take Ibuprofen/Famotidine Tablets (prepared according to formulation (A) as described above, herein referred as “HZT-501”) or identical-appearing ibuprofen 800 mg tablets thrice daily. Concomitant aspirin≦325 mg daily and anticoagulant therapies were permitted. Randomization was stratified based on aspirin/anticoagulant therapy and prior ulcer history.

Endoscopic examinations were performed during screening (baseline) and at Weeks 8, 16, and 24, with a 4-day window prior to the actual clinic visit day (the clinic visit day had a plus/minus 5-day window around the target clinic visit day). Subjects were deemed a treatment failure and terminated early from the study in the event they developed an endoscopically diagnosed UGI ulcer. Subjects who terminated early for reasons other than development of an endoscopically diagnosed UGI ulcer were to undergo an endoscopic examination at a termination visit that was conducted as soon as possible after administration of their final dose of study drug. The predefined population for primary analyses of ulcers was all subjects with ≧1 follow-up study endoscopy.

Two studies were conducted:

REDUCE-1 study group included 812 subjects; its baseline demographic parameters are provided in Table 7. For REDUCE-1 study, the primary efficacy analysis was the comparison between HZT-501 and ibuprofen of the proportion of subjects who developed endoscopically diagnosed gastric ulcers (endoscopy at 8, 16, and 24 weeks) of unequivocal depth and at least 3 mm in diameter during the 24-week treatment period. Secondary endpoints were UGI ulcers, duodenal ulcers, and serious GI complications

REDUCE-2 study group included 570 subjects; its baseline demographic parameters are provided in Table 8. For REDUCE-2 study, the primary efficacy analysis was the comparison between HZT-501 and ibuprofen of the proportion of subjects who developed endoscopically diagnosed UGI ulcers (endoscopy at 8, 16, and 24 weeks) of unequivocal depth and at least 3 mm in diameter during the 24-week treatment period. Secondary endpoints were gastric ulcers, duodenal ulcers, and serious GI complications.

TABLE 7

| | HZT-501 TID | Ibuprofen 800 mg TID |
|---|---|---|
| | (N = 607) | (N = 299) |
| Age (y), mean (range) | 55.5 (40-80) | 55.9 (40-78) |
| <65 (%) | 82.4 | 80.3 |
| ≧65 (%) | 17.6 | 19.7 |
| Women (%) | 68.7 | 69.6 |
| Race (%) | | |
| White | 76.9 | 77.9 |
| Black | 19.6 | 19.4 |
| Other | 3.5 | 3.3 |

TABLE 8

| | HZT-501 TID | Ibuprofen 800 mg TID |
|---|---|---|
| | (N = 415) | (N = 212) |
| Age (y), mean (range) | 55.3 (39-79) | 55.7 (40-79) |
| <65 (%) | 82.4 | 81.6 |
| ≧65 (%) | 17.6 | 18.4 |
| Women (%) | 65.5 | 71.7 |
| Race (%) | | |
| White | 81.4 | 82.5 |
| Black | 14.9 | 11.3 |
| Other | 3.6 | 6.1 |

REDUCE-1 study group's risk factors are shown in Table 9.

REDUCE-2 study group's risk factors are shown in Table 10.

TABLE 9
REDUCE-1: Risk Factors

| | HZT-501 TID | Ibuprofen 800 mg TID |
|---|---|---|
| | (N = 607) | (N = 299) |
| Age ≧ y (%) | 17.6 | 19.7 |
| Use of LDA and/or OAC (%) | 16.5 | 13.4 |
| Positive UGI Ulcer History (%) | 7.7 | 5.7 |
| Both (%) | 1.2 | 1.3 |

TABLE 10
REDUCE-2: Risk Factors

| | HZT-501 TID | Ibuprofen 800 mg TID |
|---|---|---|
| | (N = 415) | (N = 212) |
| Age ≧ y (%) | 17.6 | 18.4 |
| Use of LDA and/or OAC (%) | 14.7 | 13.7 |
| Positive UGI Ulcer History (%) | 5.3 | 6.1 |
| Both (%) | 0.7 | 1.4 |

B. Results (Occurrence of Gastric and/or Duodenal Ulcer)

Results of the REDUCE-1 and REDUCE-2 studies are summarized in Table 11.

TABLE 11
Proportion of Subjects with Ulcers at 24 weeks, the numbers represent
“Crude Proportions (n/N (%)); Life Table Estimates (%)”

| | REDUCE-1 | | REDUCE-2 | |
|---|---|---|---|---|
| | HZT-501 | Ibuprofen | HZT-501 | Ibuprofen |
| UGI Ulcer | 62/550 (11.3%)*; | 61/262 (23.3%)*; | 40/380 (10.5%)*; | 38/190 (20%); |
| | 14.7%* | 29.1%* | 13.8%* | 22.6% |
| Gastric Ulcer | 55/550 (10.0%)*; | 52/262 (19.8%); | 37/380 (9.7%)*; | 34/190 (17.9%); |
| | 12.9%* | 25.3% | 13.0% | 19.7% |
| Duodenal Ulcer | 7/550 (1.3%)*; | 14/262 (5.3%); | 3/380 (0.8%)*; | 9/190 (4.7%); |
| | 2.1%* | 7.1% | 0.9%* | 6.6% |
| UGI Ulcer: Subjects with | 11/89 (12.4%)*; | 10/32 (31.3%); | 8/56 (14.3%); | 6/23 (26.1%); |
| Low-dose Aspirin Use | 14.0%* | 33.3% | 14.9% | 27.5% |
| UGI Ulcer: Subjects with | 9/42 (21.4%); | 4/15 (26.7%); | 3/18 (16.7%); | 2/11 (18.2%); |
| Prior Ulcer | 17.8% | 27.3% | 23.5% | 20.0% |

UGI = upper gastrointestinal.
*P < 0.05, HZT-501 v. ibuprofen (separate comparisons for crude proportions and life table estimates.

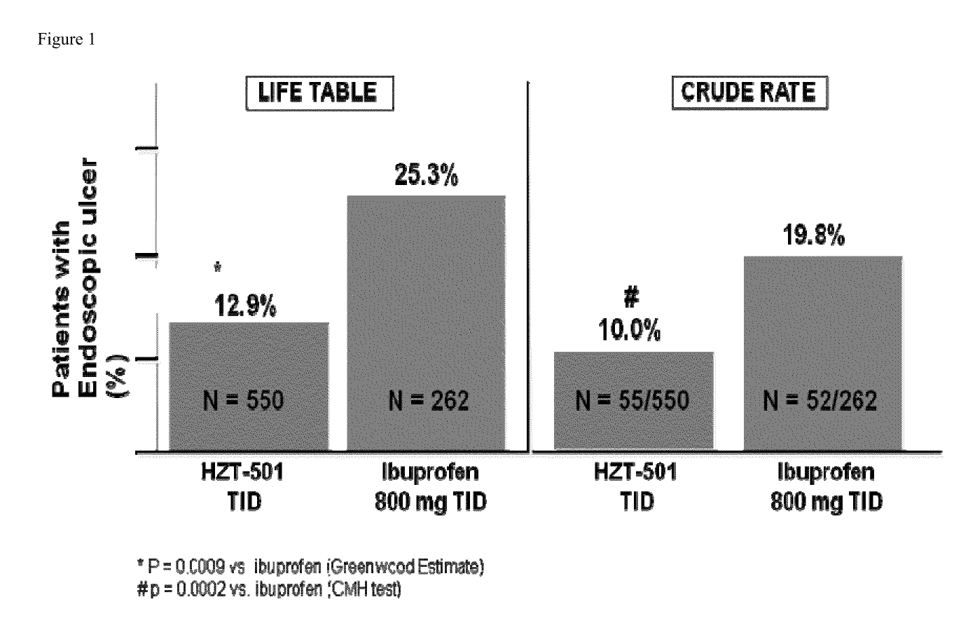

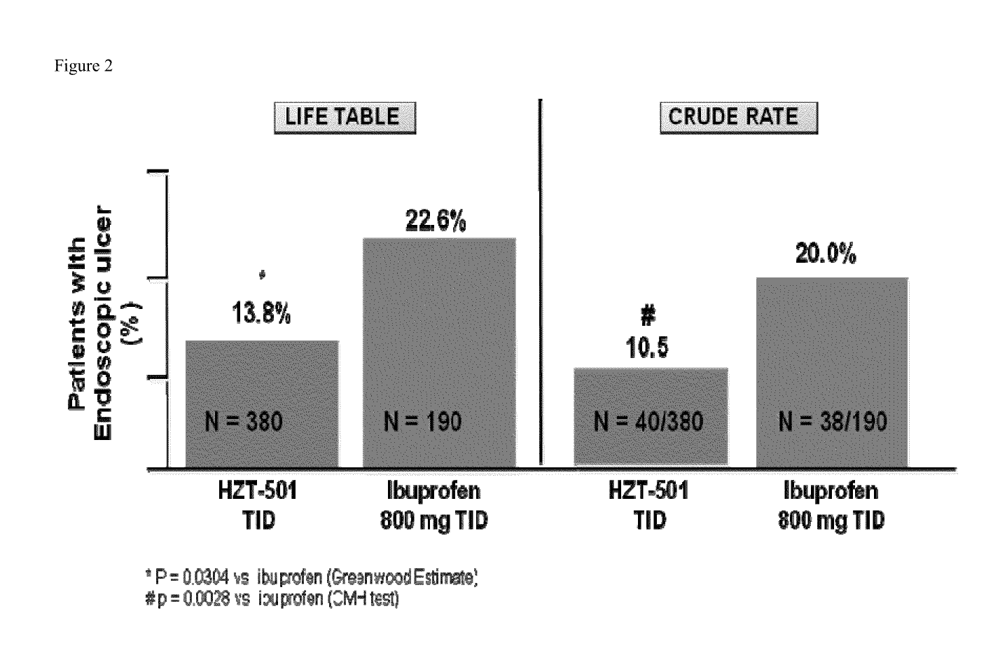

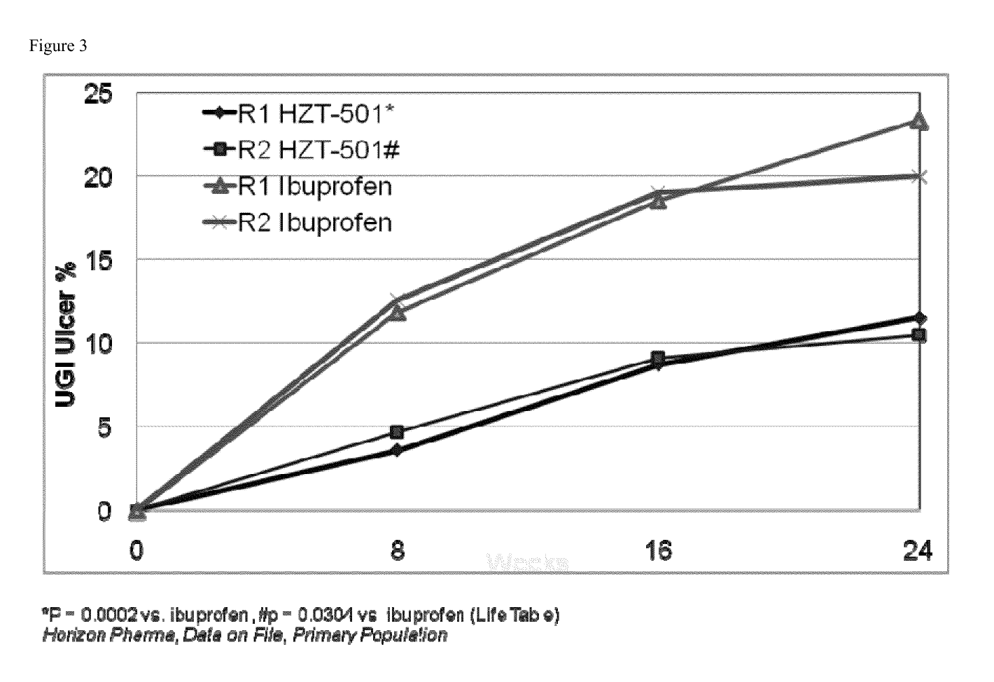

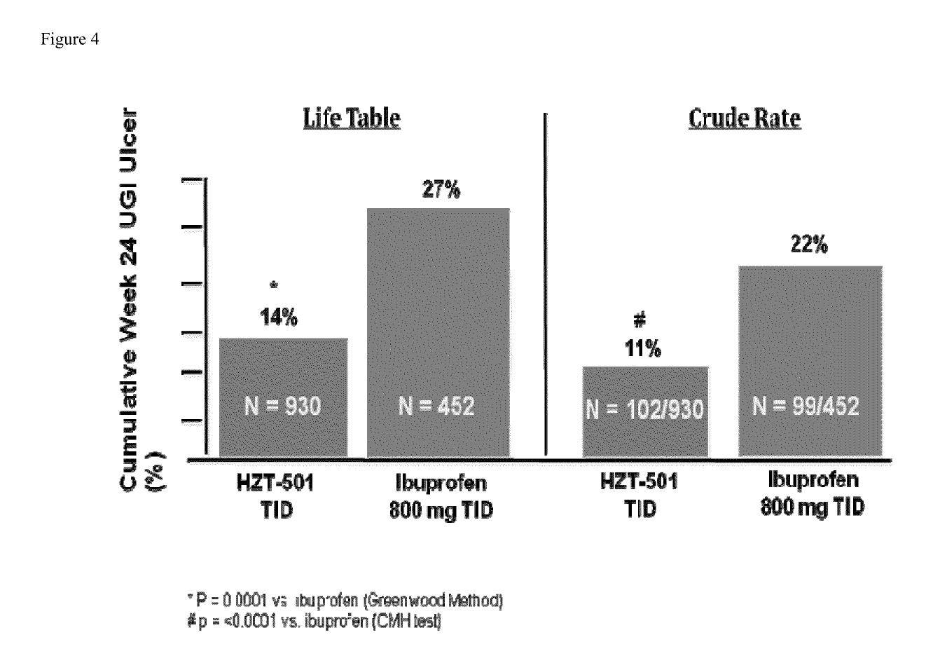

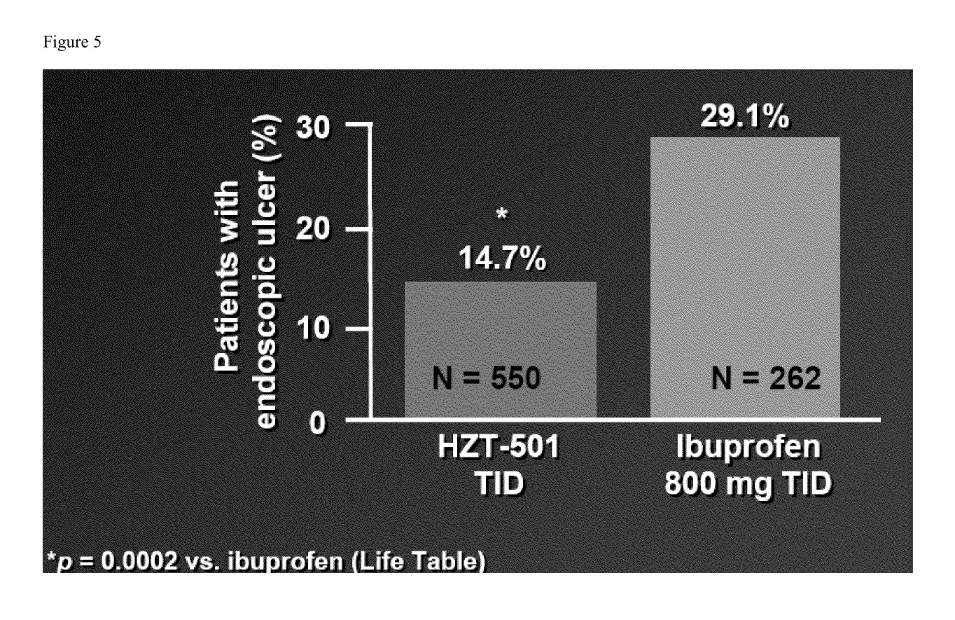

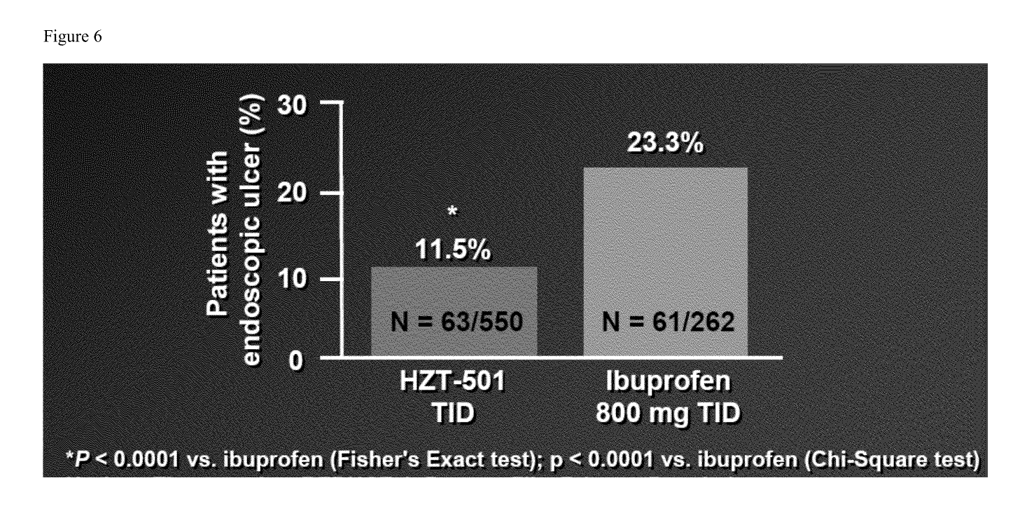

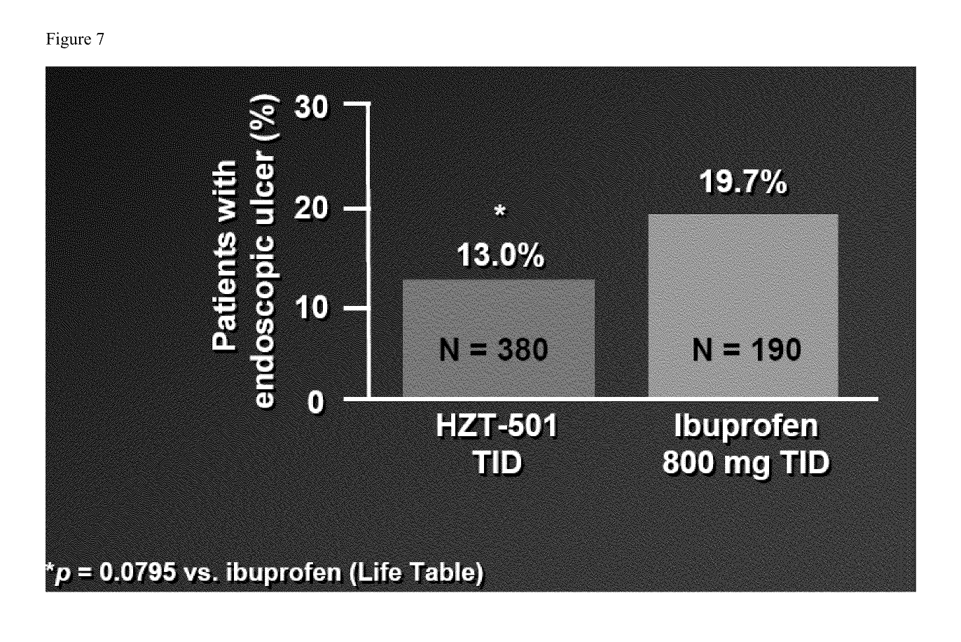

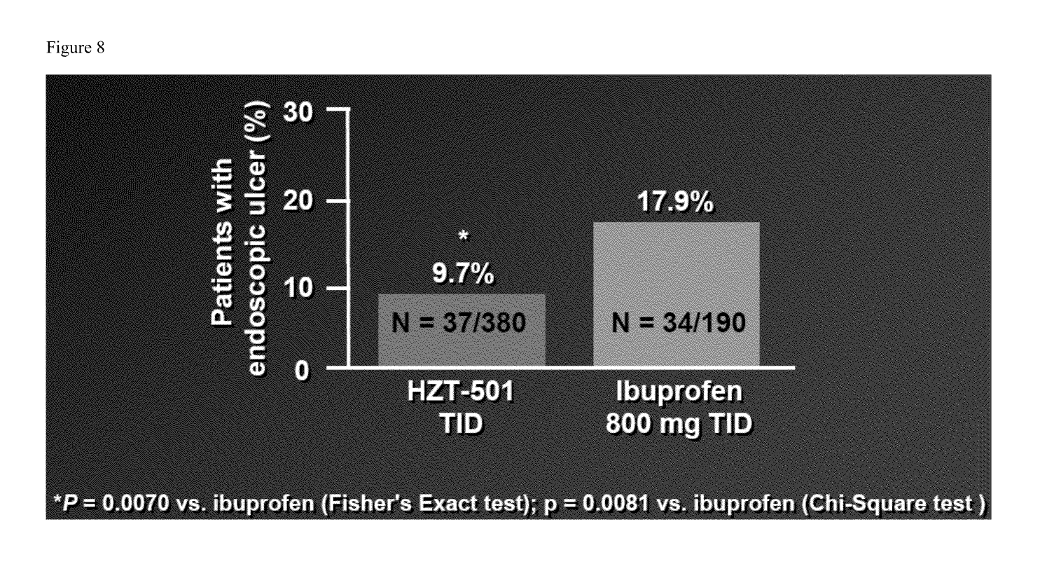

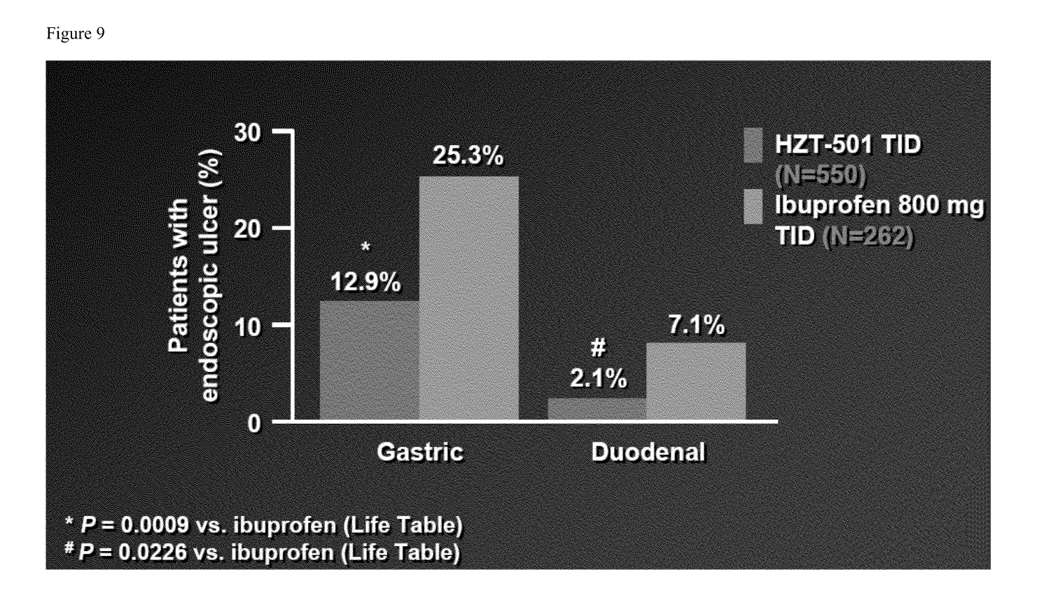

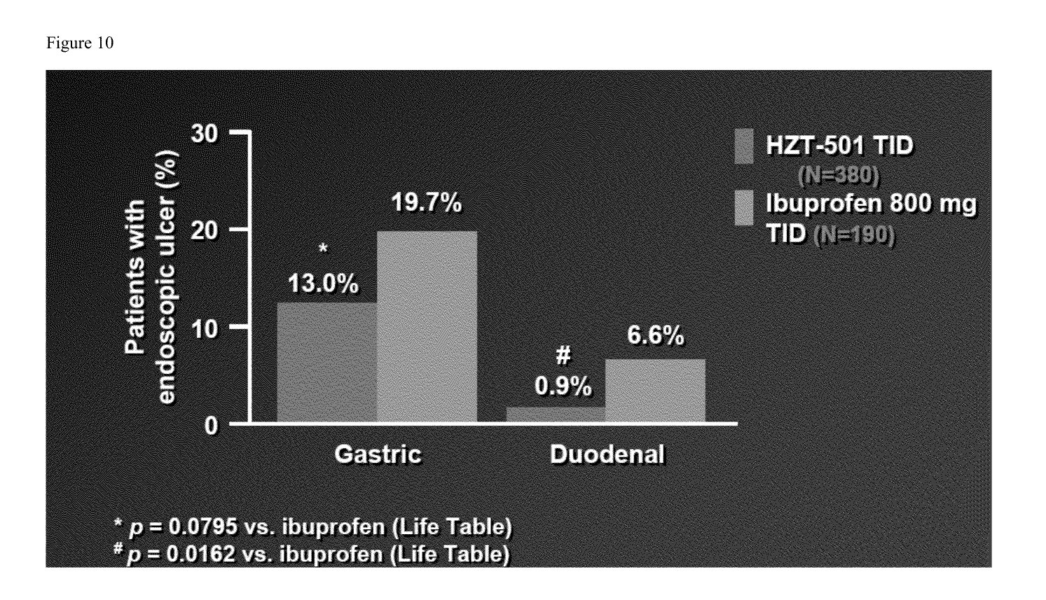

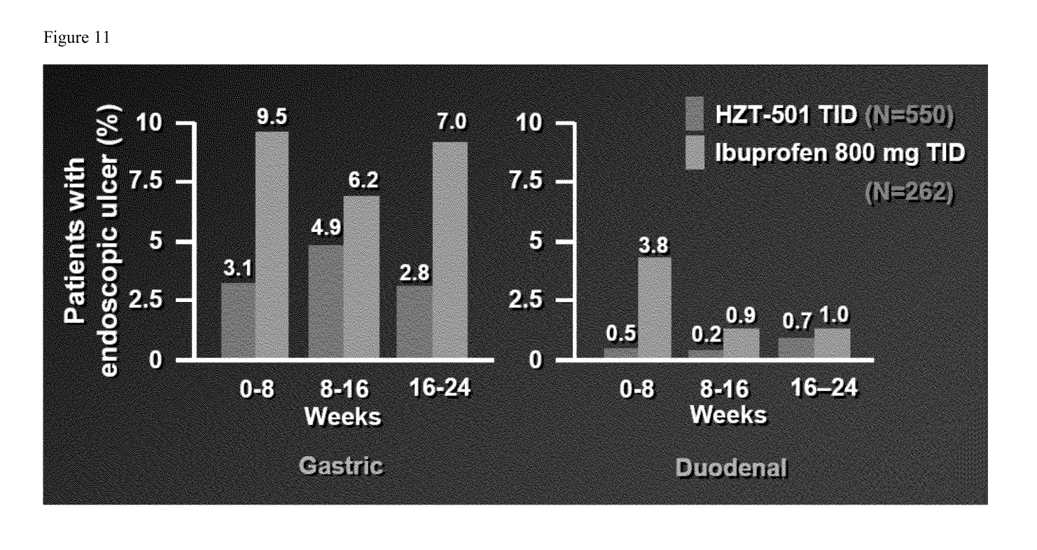

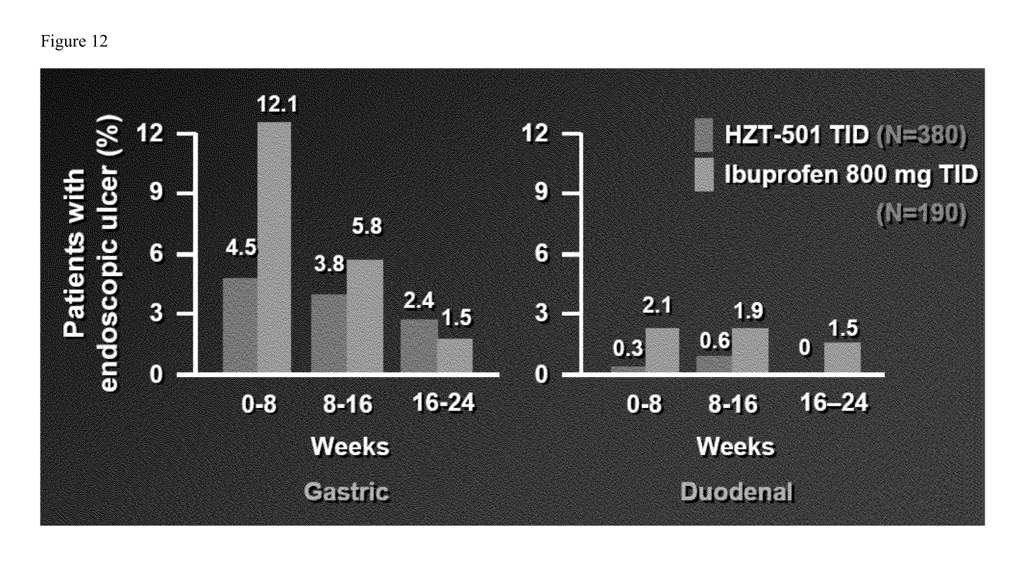

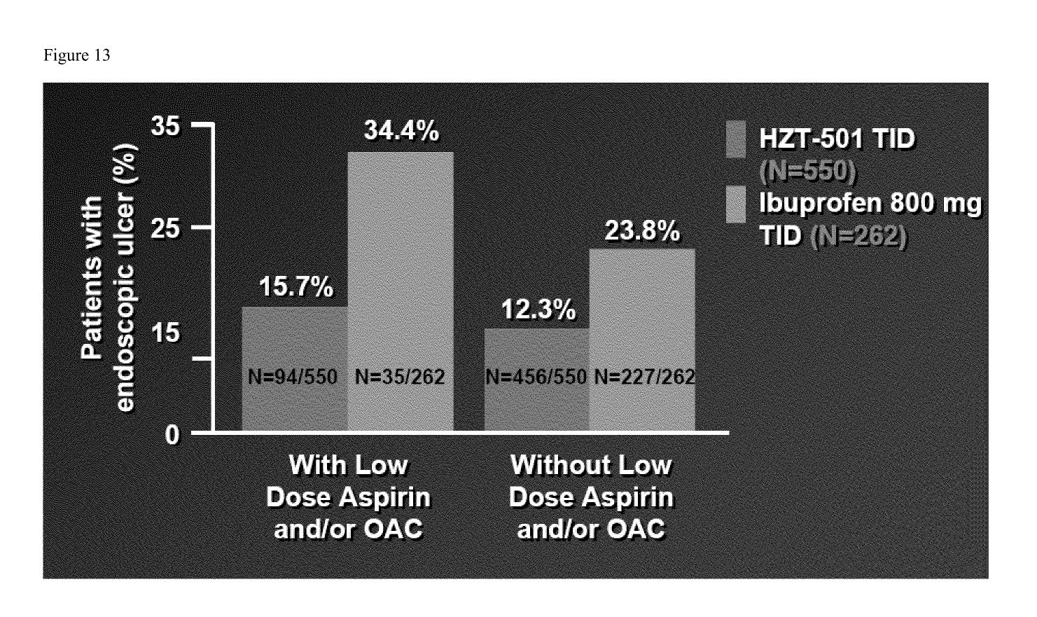

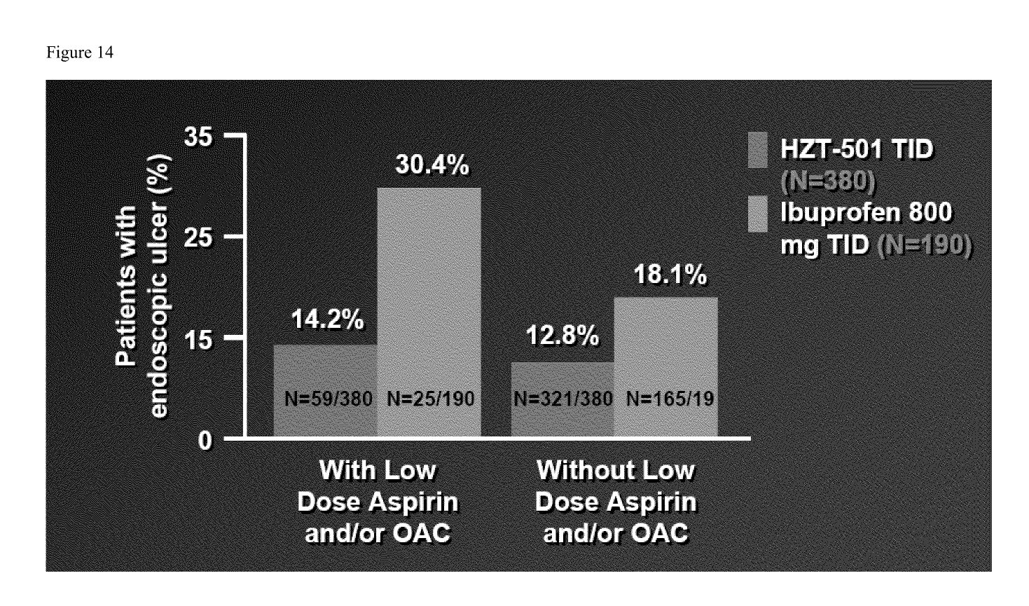

Corresponding Results are shown in FIGS. 1-14.

### C. Results (Adverse Events)

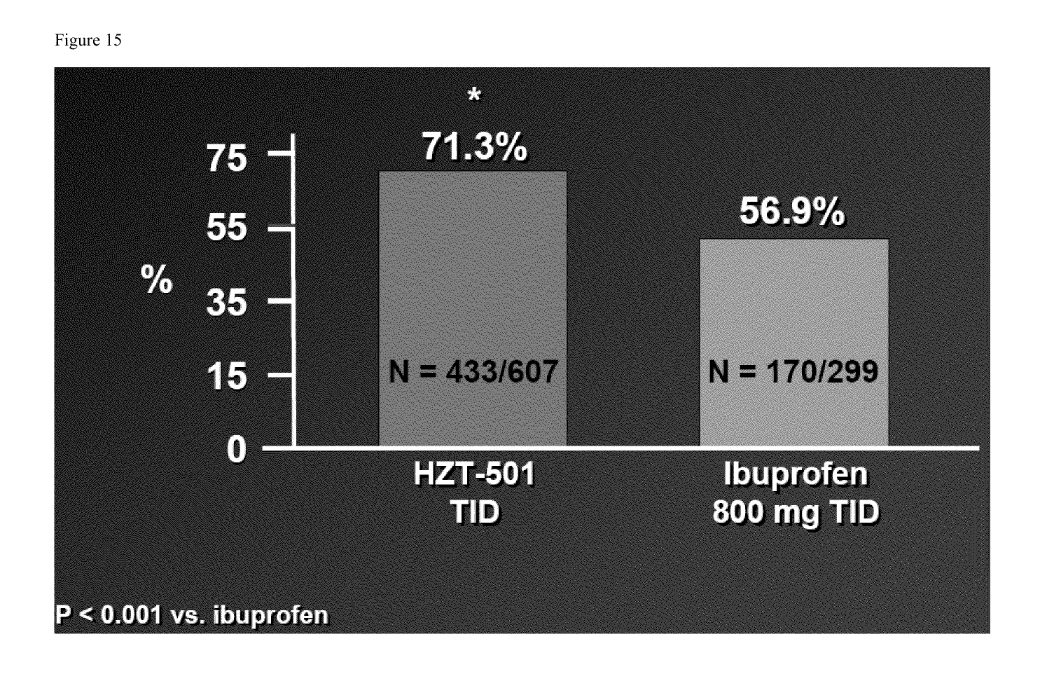

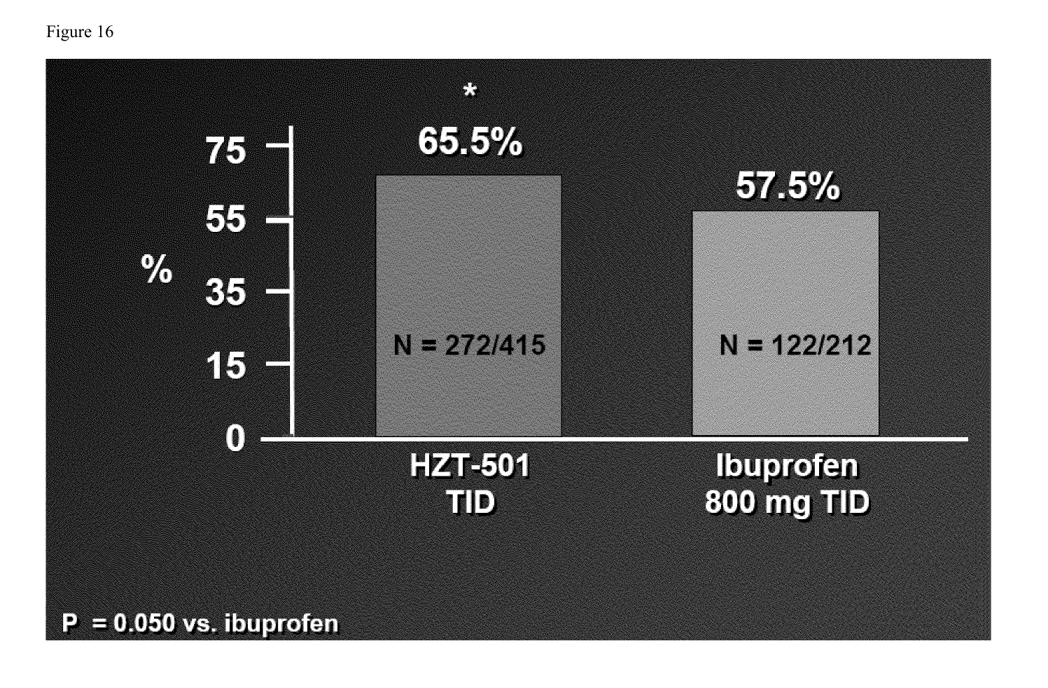

As shown in FIGS. 15 and 16, the percentage of subjects who completed the REDUCE-1 or REDUCE-2 study is higher with subjects taken HZT-501 than subjects taken ibuprofen alone.

Summary of the adverse events observed in REDUCE-1 or REDUCE-2 study group are provided in Tables 12-25:

TABLE 12
REDUCE-1: Adverse Event (“AE”) Summary

| | Incidence (%) | | |
|---|---|---|---|
| | HZT-501 TID | Ibuprofen 800 mg TID | |
| | (N = 607) | (N = 299) | |
| Subjects with at least one AE | 56.3 | 61.2 | P = 0.161 |
| AEs Leading to Discontinuation | 6.6 | 7.7 | P = 0.533 |

| | | |
|---|---|---|
| AEs by Relatedness | | |
| Possibly Related | 20.1 | 23.1 |
| Probably Not Related | 36.2 | 38.1 |
| AEs by Severity | | |
| Mild | 28.8 | 26.4 |
| Moderate | 21.4 | 27.1 |
| Severe | 5.8 | 7.0 |
| Life Threatening | 0.3 | 0.7 |

TABLE 13
REDUCE-2: Adverse Event (“AE”) Summary

| | Incidence (%) | | |
|---|---|---|---|
| | HZT-501 TID | Ibuprofen 800 mg TID | |
| | (N = 415) | (N = 212) | |
| Subjects with at least one AE | 53.0 | 54.7 | P = 0.727 |
| AEs Leading to Discontinuation | 6.7 | 7.1 | P = 0.903 |

| | | |
|---|---|---|
| AEs by Relatedness | | |
| Possibly Related | 21.4 | 27.8 |
| Probably Not Related | 31.6 | 26.9 |
| AEs by Severity | | |
| Mild | 18.1 | 26.4 |
| Moderate | 28.9 | 21.7 |
| Severe | 6.0 | 6.6 |
| Life Threatening | 0.0 | 0.0 |

TABLE 14
Integrated results of REDUCE-1 and REDUCE-2:
Adverse Event (“AE”) Summary

| | Incidence (%) | |
|---|---|---|
| | HZT-501 TID | Ibuprofen 800 mg TID |
| | (N = 1022) | (N = 511) |
| Subjects with at least one AE | 55.0 | 58.7 |
| AEs Leading to Discontinuation | 6.7 | 7.6 |
| AEs by Relatedness | | |
| Possibly Related | 20.6 | 25.0 |
| Probably Not Related | 34.3 | 33.7 |
| AEs by Severity | | |
| Mild | 24.5 | 26.4 |
| Moderate | 24.5 | 25.0 |
| Severe | 5.9 | 6.8 |
| Life Threatening | 0.3 | 0.4 |

TABLE 15
REDUCE-1: GI Adverse Events

Incidence (%)

| | HZT-501 TID | Ibuprofen 800 mg TID |
|---|---|---|
| Adverse Event | (N = 607) | (N = 299) |
| Any GI Event | 26.2 | 27.4* |
| Dyspepsia | 5.1 | 7.7 |
| Abdominal Pain Upper | 4.6 | 4.0 |
| Nausea | 6.6 | 4.3 |
| Diarrhea | 4.8 | 4.3 |
| Constipation | 4.4 | 4.3 |
| Withdrawals due to GI | 4.1 | 5.0 |

*P = 0.666

TABLE 16
REDUCE-2: GI Adverse Events

Incidence (%)

| | HZT-501 TID | Ibuprofen 800 mg TID |
|---|---|---|
| Adverse Event | (N = 415) | (N = 212) |
| Any GI Event | 25.8 | 29.7* |
| Dyspepsia | 4.1 | 8.5** |
| Abdominal Pain Upper | 1.7 | 1.9 |
| Nausea | 4.6 | 5.2 |
| Diarrhea | 4.1 | 4.2 |
| Constipation | 3.6 | 3.8 |
| Withdrawals due to GI | 3.9 | 5.2*** |

*P = 0.316
**P = 0.033
***P = 0.466

TABLE 17
REDUCE-1: Commonly Observed Adverse Events

| | Incidence (%) | |
|---|---|---|
| | HZT-501 TID | Ibuprofen 800 mg TID |
| Adverse Event | (N = 607) | (N = 299) |
| General | 5.1 | 7.7 |
| Disorders/Administrative Site | | |
| Conditions | | |
| Infections/Infestations | 20.9 | 21.2 |
| Injury, Poisoning, Procedural | 4.9 | 6.0 |
| Complications | | |
| Investigations | 4.4 | 5.0 |
| Musculoskeletal/connective | 8.1 | 8.0 |
| Tissue Disorders | | |
| Nervous System Disorders | 7.9 | 7.0 |
| Respiratory, Thoracic and | 7.1 | 8.7 |
| Mediastinal Disorders | | |

TABLE 18
REDUCE-2: Commonly Observed Adverse Events

| | Incidence (%) | |
|---|---|---|
| | HZT-501 TID | Ibuprofen 800 mg TID |
| Adverse Event | (N = 415) | (N = 212) |
| General | 4.8 | 3.8 |
| Disorders/Administrative Site | | |
| Conditions | | |
| Infections/Infestations | 20.2 | 19.3 |
| Injury, Poisoning, Procedural | 4.3 | 3.8 |
| Complications | | |
| Investigations | 4.6 | 2.4 |
| Musculoskeletal/connective | 7.5 | 5.2 |
| Tissue Disorders | | |
| Nervous System Disorders | 6.3 | 4.7 |
| Respiratory, Thoracic and | 8.0 | 4.7 |
| Mediastinal Disorders | | |

TABLE 19
REDUCE-1: Adverse Events of Interest

| | Incidence (%) | |
|---|---|---|
| | HZT-501 TID | Ibuprofen 800 mg TID |
| Adverse Event | (N = 607) | (N = 299) |
| Renal and Urinary Disorders | 1.8 | 1.0 |
| Cardiac Disorders | 0.7 | 1.3 |
| Hepatobiliary Disorders | 0.5 | 0.0 |
| Hypertension | 3.5 | 2.3 |

TABLE 20
REDUCE-2: Adverse Events of Interest

| | Incidence (%) | |
|---|---|---|
| | HZT-501 TID | Ibuprofen 800 mg TID |
| Adverse Event | (N = 415) | (N = 212) |
| Renal and Urinary Disorders | 1.7 | 0.0 |
| Cardiac Disorders | 0.0 | 0.5 |
| Hepatobiliary Disorders | 0.2 | 0.0 |
| Hypertension | 2.7 | 1.9 |

TABLE 21
Integrated results of REDUCE-1 and REDUCE-2: treatment-emergent
Adverse Events occurring in >1% of the safety population

| | HZT-501 | Ibuprofen | Total | |
|---|---|---|---|---|
| System Organ Class | N = 1022 | N = 511 | N = 1533 | |
| Preferred Term | % (n) | % (n) | % (n) | P-value* |
| Total Subjects With at Least One TEAE | 55.0 (562) | 58.7 (300) | 56.2 (862) | 0.1620 |
| Blood and lymphatic system disorders | 1.9 (19) | 1.8 (9) | 1.8 (28) | 0.8939 |
| Anaemia | 1.6 (16) | 1.4 (7) | 1.5 (23) | 0.7703 |
| Gastrointestinal disorders | 26.0 (266) | 28.4 (145) | 26.8 (411) | 0.3291 |
| Dyspepsia | 4.7 (48) | 8.0 (41) | 5.8 (89) | 0.0086 |
| Nausea | 5.8 (59) | 4.7 (24) | 5.4 (83) | 0.3842 |
| Diarrhoea | 4.6 (47) | 4.3 (22) | 4.5 (69) | 0.7975 |
| Constipation | 4.1 (42) | 4.1 (21) | 4.1 (63) | 0.9951 |
| Abdominal pain upper | 3.3 (34) | 2.5 (13) | 3.1 (47) | 0.4012 |
| Gastro-oesophageal reflux disease | 1.9 (19) | 3.1 (16) | 2.3 (35) | 0.1181 |
| Vomiting | 2.3 (23) | 1.8 (9) | 2.1 (32) | 0.5355 |
| Stomach discomfort | 1.8 (18) | 1.6 (8) | 1.7 (26) | 0.7865 |
| Abdominal pain | 1.5 (15) | 1.6 (8) | 1.5 (23) | 0.8865 |
| Gastritis | 1.0 (10) | 1.4 (7) | 1.1 (17) | 0.4988 |
| Abdominal distension | 0.9 (9) | 1.4 (7) | 1.0 (16) | 0.3757 |
| Flatulence | 1.4 (14) | 0.4 (2) | 1.0 (16) | 0.0761 |
| Abdominal tenderness | 0.8 (8) | 1.0 (5) | 0.8 (13) | |
| General disorders and administration site | 5.0 (15) | 4.5 (23) | 4.8 (74) | 0.6770 |
| conditions | | | | |
| Oedema peripheral | 2.0 (20) | 1.8 (9) | 1.9 (29) | 0.7979 |
| Fatigue | 0.5 (5) | 1.0 (5) | 0.7 (10) | |
| Infections and infestations | 20.6 (211) | 20.7 (106) | 20.7 (317) | 0.9604 |
| Upper respiratory tract infection | 3.8 (39) | 4.1 (21) | 3.9 (60) | 0.7844 |
| Nasopharyngitis | 2.4 (25) | 2.7 (14) | 2.5 (39) | 0.7374 |
| Sinusitis | 2.1 (21) | 2.7 (14) | 2.3 (35) | 0.3936 |
| Bronchitis | 2.4 (25) | 1.2 (6) | 2.0 (31) | 0.0948 |
| Urinary tract infection | 1.7 (17) | 2.3 (12) | 1.9 (29) | 0.3426 |
| Influenza | 1.7 (17) | 1.6 (8) | 1.6 (25) | 0.8900 |
| Gastroenteritis viral | 1.0 (10) | 0.8 (4) | 0.9 (14) | |
| Injury, poisoning and procedural complications | 4.7 (48) | 5.1 (26) | 4.8 (74) | 0.7295 |
| Fall | 0.8 (8) | 1.0 (5) | 0.8 (13) | |
| Musculoskeletal and connective tissue disorders | 7.8 (80) | 6.8 (35) | 7.5 (115) | 0.4983 |
| Arthralgia | 1.3 (13) | 1.8 (9) | 1.4 (22) | 0.4382 |
| Back pain | 1.8 (18) | 0.8 (4) | 1.4 (22) | 0.1255 |
| Pain in extremity | 1.4 (14) | 0.8 (4) | 1.2 (18) | 0.3136 |
| Musculoskeletal pain | 1.0 (10) | 0.8 (4) | 0.9 (14) | |
| Muscle spasms | 1.0 (10) | 0.4 (2) | 0.8 (12) | |
| Nervous system disorders | 7.2 (74) | 6.1 (31) | 6.8 (105) | 0.3974 |
| Headache | 3.3 (34) | 3.3 (17) | 3.3 (51) | 0.9964 |
| Dizziness | 1.2 (12) | 1.4 (7) | 1.2 (19) | 0.7356 |
| Psychiatric disorders | 2.8 (29) | 3.1 (16) | 2.9 (45) | 0.7555 |
| Insomnia | 1.1 (11) | 1.0 (5) | 1.0 (16) | 0.8542 |
| Respiratory, thoracic and mediastinal disorders | 7.4 (76) | 7.0 (36) | 7.3 (112) | 0.785 |
| Cough | 1.9 (19) | 2.0 (10) | 1.9 (29) | 0.8846 |
| Pharyngolaryngeal pain | 1.6 (16) | 1.2 (6) | 1.4 (22) | 0.5415 |
| Sinus congestion | 0.6 (6) | 1.4 (7) | 0.8 (13) | 0.1162 |
| Dyspnoea | 0.6 (6) | 1.0 (5) | 0.7 (11) | |
| Skin and subcutaneous tissue disorders | 2.9 (30) | 3.5 (18) | 3.1 (48) | 0.5364 |
| Rash | 0.5 (5) | 1.0 (5) | 0.7 (10) | |
| Vascular disorders | 3.7 (38) | 2.7 (14) | 3.4 (52) | 0.3223 |
| Hypertension | 3.1 (32) | 2.2 (11) | 2.8 (43) | 0.2771 |

TEAE = treatment-emergent adverse events
All adverse events were coded using the Medical Dictionary for Regulatory Activities (MedDRA) dictionary (Version 9.1). A subject with multiple events per System Organ Class or per Preferred Term category is counted only once per subject.
*P-value for the integrated data is from a Cochran-Mantel-Haenszel test for adverse events occurring in ≧1% of subjects, controlling for study.

TABLE 22
REDUCE-1: Hepatic Effects

| | Number of Subjects | |
|---|---|---|
| | HZT-501 TID | Ibuprofen 800 mg TID |
| Shift from Normal to High* | (N = 607) | (N = 299) |

AST

| | | |
|---|---|---|
| 8 weeks | 3 | 2 |
| 16 weeks | 0 | 1 |
| 24 weeks | 2 | 2 |

ALT

| | | |
|---|---|---|
| 8 weeks | 4 | 1 |
| 16 weeks | 0 | 1 |
| 24 weeks | 2 | 1 |

AST = aspartate aminotransferase; ALT = alanine aminotransferase
*from baseline to the study week indicated.

TABLE 23
REDUCE-2: Hepatic Effects

| | Number of Subjects | |
|---|---|---|
| | HZT-501 TID | Ibuprofen 800 mg TID |
| Shift from Normal to High* | (N = 415) | (N = 212) |

AST

| | | |
|---|---|---|
| 8 weeks | 1 | 0 |
| 16 weeks | 1 | 0 |
| 24 weeks | 2 | 0 |

ALT

| | | |
|---|---|---|
| 8 weeks | 0 | 0 |
| 16 weeks | 0 | 0 |
| 24 weeks | 1 | 0 |

AST = aspartate aminotransferase; ALT = alanine aminotransferase
*from baseline to the study week indicated.

TABLE 24
REDUCE-1: Adverse Events and Death

| | Number/Incidence % | |
|---|---|---|
| | HZT-501 TID | Ibuprofen 800 mg TID |
| | (N = 607) | (N = 299) |
| Total SAEs | 21/3.5% | 13/4.3% |
| SAEs leading to discontinuation | 6/1.0% | 2/0.7% |
| Deaths | 0/0.0% | 1/0.1% |

TABLE 25
REDUCE-2: Adverse Events and Death

| | Number/Incidence % | |
|---|---|---|
| | HZT-501 TID | Ibuprofen 800 mg TID |
| | (N = 415) | (N = 212) |
| Total SAEs | 11/2.7% | 4/1.9% |
| SAEs leading to discontinuation | 2/0.5% | 2/0.9% |
| Deaths | 0/0.0% | 0/0.0% |

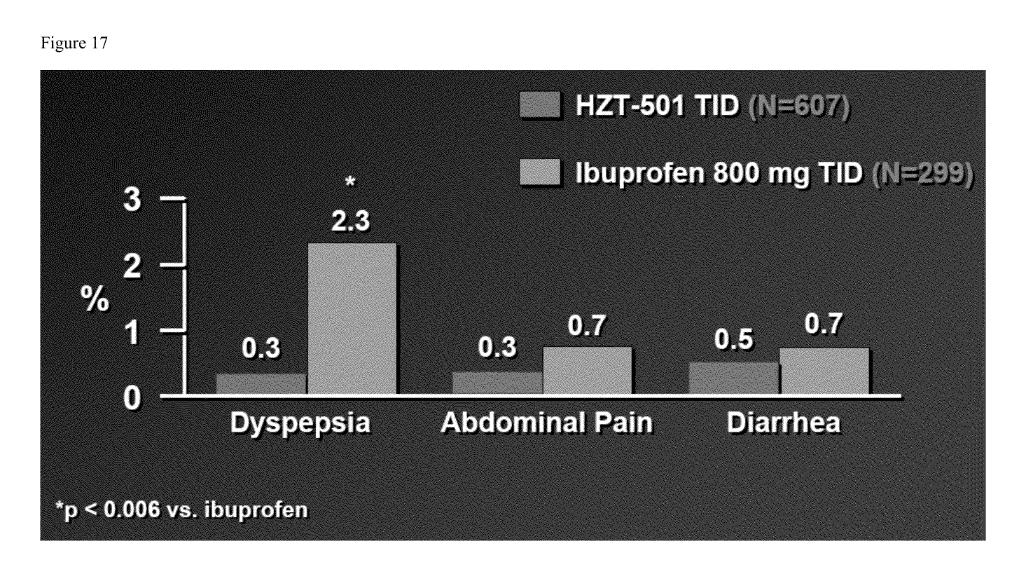

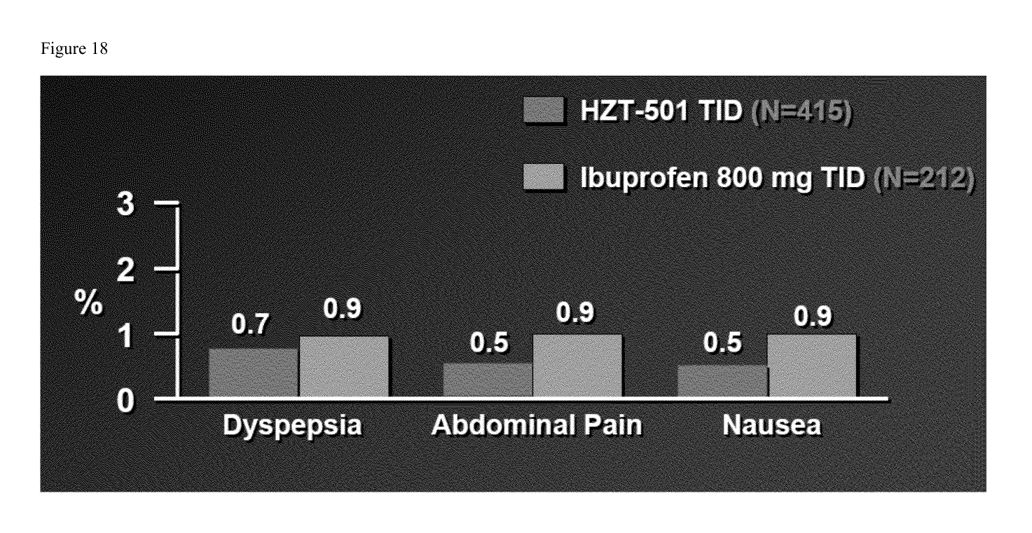

Discontinuation rates due to gastrointestinal adverse events for HZT-501 and Ibuprofen are shown in FIG. 17 (REDUCE-1) and FIG. 18 (REDUCE-2).

The primary efficacy measurement for both REDUCE-1 and REDUCE-2 studies was endoscopic examination for the presence of an UGI ulcer in REDUCE-1 and the presence of a gastric ulcer in REDUCE-2. Ulcers were defined as endoscopically diagnosed ulcers of unequivocal depth and at least 3 mm in diameter. It is accepted clinically that UGI ulcers of unequivocal depth and at least 3 mm in diameter can be evaluated reproducibly by different clinical investigators. Subjects who develop UGI ulcers are known to be at risk for developing serious GI complications such as perforation of ulcers, gastric outlet obstruction due to ulcers, and GI bleeding. A clinically accepted standard of care for subjects who present with potential signs and symptoms consistent with UGI ulcer is an endoscopic examination to determine whether ulceration is present.

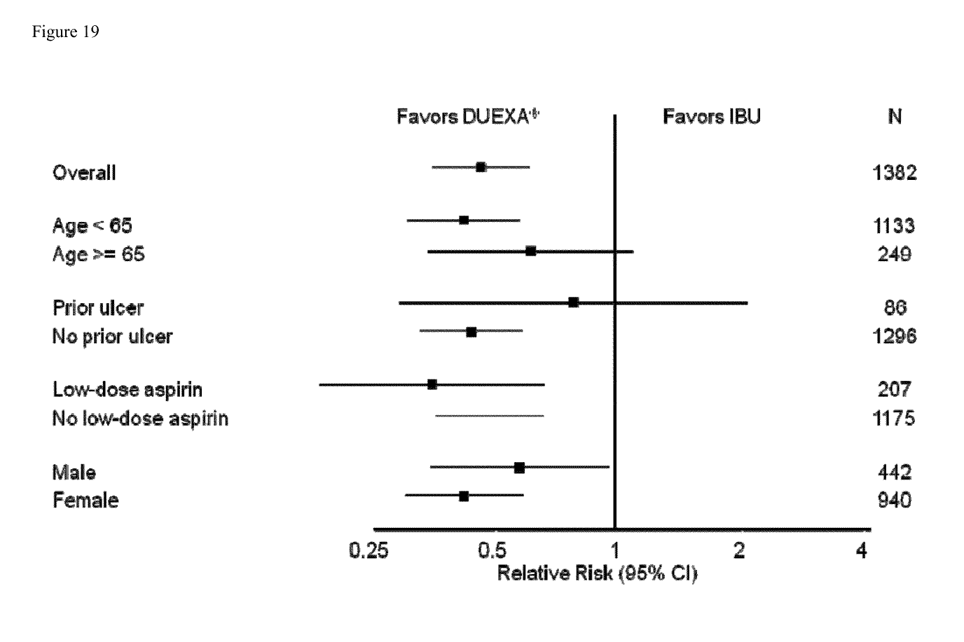

In addition to examining potential risk factors together in a statistical model, the effect of treatment was examined in the combined studies overall and within various subgroups of interest. These subgroups included age category (<65, ≧55), prior history of UGI ulcer (yes, no), use of low-dose aspirin (LDA) (yes, no), and gender. The relative risks and their 95% CI for UGI ulcers of the treatment effect of HZT-501 versus ibuprofen for each subgroup population were derived with only the subjects in that subgroup population included in the proportional hazards regression model. Treatment was the only factor included in the model. The proportion of subjects in the primary population who developed at least one UGI ulcer by demographic group (i.e., age, race, and gender) for the pooled data from the primary populations from Studies REDUCE-1 and REDUCE-2 is shown in Table 26. The forest plot of these overall results as well as those for each subgroup is shown in FIG. 19. A relative risk<1.0 favors HZT-501 over ibuprofen alone.

The findings from the subgroup analyses are consistent with the overall results in supporting a relative risk reduction with HZT-501 compared to ibuprofen alone regardless of age, prior ulcer history, use of LDA, or gender even though the studies were not powered for a specific subgroup.

TABLE 26:
Proportion of Subjects who Developed at Least One Upper Gastrointestinal
Ulcer by Demographic Strata (Primary Population) - Pooled
Data from Studies REDUCE-1 and REDUCE-2

| | HZT-501 | Ibuprofen | | |
|---|---|---|---|---|
| | (N = 930) | (N = 452) | Difference | |
| Demographic | Proportion | Proportion | Proportion | |
| Strata | (95% CI)^{a} | (95% CI)^{a} | (95% CI) | P-value^{b} |
| Age class | | | | |
| <65 years | 12.4% | 26.3% | 13.9% | <0.0001 |
| | (9.9%, 15.7%) | (21.4%, 32.2%) | (7.8%, 20.0%) | |
| ≧65 years | 22.9% | 26.6% | 3.7% | 0.5983 |
| | (16.0%, 32.1%) | (17.3%, 39.5%) | (−10.0%, 17.3%) | |
| Race | | | | |
| White | 15.4% | 27.8% | 12.5% | 0.0001 |
| | (12.4%, 18.9%) | (22.7%, 33.8%) | (6.0%, 18.9%) | |
| Black or | 10.9% | 22.5% | 11.6% | 0.0650 |
| African-American | (6.3%, 18.4%) | (13.7%, 35.8%) | (−0.7%, 24.0%) | |
| Other^{c} | 7.1% | 13.3% | 6.2% | 0.5382 |
| | (1.8%, 25.8%) | (3.5%, 43.6%) | (−13.5%, 25.9%) | |
| Gender | | | | |
| Male | 16.5% | 22.2% | 5.6% | 0.2540 |
| | (11.9%, 22.8%) | (15.3%, 31.5%) | (−4.0%, 15.3%) | |
| Female | 13.3% | 28.3% | 15.0% | <0.0001 |
| | (10.4%, 16.9%) | (22.8%, 34.9%) | (8.2%, 21.8%) | |

Source: ISE Table 3.1.1.2
CI = confidence interval; ISE = Integrated Summary of Efficacy
^{a}Week 24 proportions are estimated from a life table analysis that included a covariate for treatment.
^{b}P-value is for the difference of the Week 24 estimated proportion of subjects developing at least one ulcer.
^{c}“Other” class includes the following races: Native Hawaiian or other Pacific Islander, Asian, American Indian or Alaska Native, and races reported as “Other.”

### D. Summary

1. Conclusions from Reduce-2

Efficacy results and conclusions for the a priori specified life table method for the primary population are summarized as follows:
Study met the prespecified primary efficacy objective and demonstrated a statistically significant reduction in the proportion of subjects who developed at least one UGI ulcer in the HZT-501 group (13.8%) compared to the ibuprofen group at Week 24 (22.6%; P=0.0304) in the life table analysis. This study showed nearly a 40% reduction in UGI ulcer rate when HZT-501 is taken compared to ibuprofen alone.
The primary analysis result for the primary efficacy objective is supported by the sensitivity analysis of crude incidence rate for subjects who developed at least one UGI ulcer; the crude incidence rate analysis demonstrated a statistically significant reduction of approximately 50% in the incidence of UGI ulcers in the HZT-501 group (10.5%) compared to the ibuprofen group at Week 24 (20.0%; P=0.0028). Results for the highly conservative sensitivity analysis of crude ulcer rate with early drop-outs imputed as treatment failures showed a statistically significant reduction in crude ulcer rates for HZT-501 compared to ibuprofen alone for UGI ulcers (17.6% vs. 25.3%; P=0.0357).
The primary analysis results in the life table analysis for the two secondary efficacy objectives of ulcer reduction demonstrated a reduction in the proportion of subjects who developed at least one gastric ulcer in the HZT-501 group (13.0%) compared to the ibuprofen group (19.7%; P=0.0795) and a reduction in the proportion of subjects who developed at least one duodenal ulcer in the HZT-501 group (0.9%) compared to the ibuprofen group (6.6%). The duodenal ulcer comparison was not formally tested statistically due to the hierarchical testing rule for the efficacy outcomes. The direction of effect is supportive of the advantageous effect of HZT-501 over ibuprofen on the reduction of ibuprofen-induced ulcers.
Subjects receiving HZT-501 demonstrated a significant reduction in both secondary ulcer outcome measures compared to those receiving ibuprofen using the crude incidence rate sensitivity analysis. The overall incidence of gastric ulcers was significantly reduced in the HZT-501 group (9.7%) as compared to the ibuprofen group (17.9%; P=0.0070). The overall incidence for duodenal ulcers also was significantly reduced in the HZT-501 group (0.8%) compared to the ibuprofen group (4.7%; P=0.0035). Due to the relative increase in ulcer rates when subjects who terminated early and met the revised rule are imputed as having an ulcer, statistical significance was not strictly achieved for the secondary endpoint of gastric ulcer based on the 0.05 threshold (17.1% vs. 23.2%, P=0.0906) for subjects receiving HZT-501 compared to ibuprofen alone. These crude rate results support the overall conclusions of the primary analysis method showing that the addition of famotidine to ibuprofen reduces the incidence of UGI ulceration associated with ibuprofen use. For the secondary endpoint of duodenal ulcer, there were so few endoscopically diagnosed duodenal ulcers (three in the HZT-501 group and nine in the ibuprofen group) that when subjects imputed as ulcers under the revised rule are added to the ulcer totals, the original treatment difference is negated (8.2% vs. 10.1%, P=0.5300; for subjects receiving HZT-501 compared to ibuprofen alone).
No subjects developed NSAID-associated serious GI complications (a secondary efficacy objective) during this study.
The incidences of UGI ulcers, gastric ulcers, and duodenal ulcers were numerically lower in the HZT-501 group compared to the ibuprofen group at Week 8, 16, and 24 (except for gastric ulcers), and overall
   Conclusions from Reduce-1

Efficacy results and conclusions for the a priori specified life table method for the primary population are summarized as follows:
Study met the prespecified primary efficacy objective and demonstrated a statistically significant reduction in the proportion of subjects who developed at least one gastric ulcer in the HZT-501 group (12.9%) compared to the ibuprofen group at Week 24 (25.3%; P=0.0009) in the life table analysis. This study showed nearly a 50% reduction in ulcer rate when HZT-501 is taken compared to ibuprofen alone.
The primary analysis result for the primary efficacy objective is supported by the sensitivity analysis of crude incidence rate for subjects who developed at least one gastric ulcer; the crude incidence rate demonstrated a statistically significant reduction of approximately 50% in the incidence of gastric ulcers in the HZT-501 group (10.0%) compared to the ibuprofen group (19.8%; P=0.0002). Results for the highly conservative sensitivity analysis of crude ulcer rate with early drop-outs imputed as treatment failures showed a statistically significant reduction in crude ulcer rates for HZT-501 compared to ibuprofen alone for gastric ulcers (13.8% vs. 26.7%; P<0.0001).
Both prespecified secondary ulcer reduction objectives were met in the life table analysis. The subjects receiving HZT-501 experienced a statistically significant reduction in the proportion of subjects who developed at least one UGI ulcer (14.7%) compared to the ibuprofen group (29.1%; P=0.0002). A statistically significant reduction in the proportion of subjects who developed at least one duodenal ulcer also was observed in the HZT-501 group (2.1%) compared to the ibuprofen group (7.1%; P=0.0226).
Subjects receiving HZT-501 demonstrated a significant reduction in both secondary ulcer outcome measures compared to those receiving ibuprofen using the crude incidence rate sensitivity analysis. The overall incidence of UGI ulcers was significantly reduced in the HZT-501 group (11.3%) as compared to the ibuprofen group (23.3%; P<0.0001). The overall incidence of duodenal ulcers also was significantly reduced in the HZT-501 group (1.3%) compared to the ibuprofen group (5.3%; P=0.0014). The second sensitivity analyses of crude ulcer rate with early drop-outs imputed as treatment failures showed a highly statistically significant reduction in crude ulcer rates for UGI ulcer (15.1% vs. 30.2%; P<0.0001) and duodenal ulcers (5.5% vs. 12.6%: P=0.0007) for subjects receiving HZT-501 compared to ibuprofen alone. These crude rate results support the overall conclusions of the primary analysis method showing that the addition of famotidine to ibuprofen reduces the incidence of UGI ulceration associated with ibuprofen use.
A secondary efficacy endpoint of NSAID associated serious GI complications was reported in 0.6% of subjects in the HZT-501 group and in none of the subjects in the ibuprofen group (P=0.0824). There was a low incidence of serious GI complications and the clinical significance of GI bleeding only observed endoscopically in three subjects at a single study site is uncertain. As with other gastroprotective strategies, GI bleeding may still occur in any subject taking NSAID-containing products, and clinical surveillance for signs and symptoms of GI bleeding is indicated in such subjects.
The incidences of gastric ulcers, UGI ulcers, and duodenal ulcers were numerically lower in the HZT-501 group compared to the ibuprofen group at Week 8, 16, 24, and overall.

Based on the foregoing results, it is concluded that:

(1) HZT-501 result in a statistically and clinically significant reduction in the incidence of NSAID-induced gastric and/or duodenal ulcers;

(2) HZT-501 result in a similar reduction in the incidence of NSAID-induced ulcers in subjects taking low-dose aspirin or were positive Hx of peptic ulcer disease;

(3) HZT-501 led to significantly fewer GI ulcers compared to ibuprofen alone within 8 weeks of initiating treatment;

(4) HZT-501's overall safety profile was similar to that of ibuprofen, no unexpected, additive or synergistic toxicities between the two component drugs;

(5) More subjects completed HZT-501 therapy versus ibuprofen; and

(6) Fewer subjects withdraw from the studies due to dyspepsia with HZT-501 than ibuprofen.

### III. Follow-On Safety Study of HZT-501 in Subjects Who Have Completed Participation in REDUCE-1 or REDUCE-2

### A. Method

Subjects who did not develop an endoscopically-diagnosed upper gastrointestinal (i.e., gastric and/or duodenal) ulcer after the completion of the 24-week REDUCE-1 or REDUCE-2 study were enrolled in this follow-on safety study within 1 week of completion of REDUCE-1 or REDUCE-2 study.

Subjects enrolled in this follow-on safety study retained their treatment assignment from the contributing study, and they were required to take HZT-501 or ibuprofen for another 28 weeks. More subjects who had taken HZT-50 during REDUCE-1 or REDUCE-2 study enrolled in this follow-on safety study than ibuprofen. (approximately 3:1 HZT-501/Ibuprofen were enrolled).

Baseline demographic parameters of the enrolled subjects are provided in Table 27.

TABLE 27

| | HZT-501 TID | Ibuprofen 800 mg TID |
|---|---|---|
| | (N = 132) | (N = 47) |
| Age (y), mean (range) | 55.7 (41-79) | 53.6 (41-75) |
| <65 (%) | 83.3 | 87.2 |
| ≧65 (%) | 16.7 | 12.8 |
| Women (%) | 68.2 | 68.1 |
| Race (%) | | |
| white | 90.2 | 89.4 |
| Black | 6.8 | 4.3 |
| Other | 3.0 | 6.4 |
| Contributing Study (%) | | |
| Reduce -2 | 35.6 | 31.9 |
| Reduce-1 | 64.4 | 68.1 |

The risk factors of the studied group are summarized in Table 28.

TABLE 28

| | HZT-501 TID | Ibuprofen 800 mg TID |
|---|---|---|
| | (N = 132) | (N = 47) |
| Age ≧ y (%) | 16.7 | 12.8 |
| Use of LDA and/or OAC (%) | 15.2 | 14.9 |
| Positive UGI Ulcer History (%) | 5.3 | 2.1 |

### B. Results

The percentage of subjects who completed this follow-on study is shown in FIG. 19.

Summary of adverse events observed in the follow-on study group are provided in Tables 29-35:

TABLE 29
Follow-on Study: Adverse Event (“AE”) Summary

| | Incidence (%) | | |
|---|---|---|---|
| | HZT-501 TID | Ibuprofen 800 mg TID | |
| | (N = 132) | (N = 47) | |
| Subjects with at least one AE | 41.7 | 34.0 | P = 0.423 |
| AEs Leading to Discontinuation | 1.5 | 2.1 | |

| | | |
|---|---|---|
| AEs by Relatedness | | |
| Possibly Related | 4.5 | 8.5 |
| Probably Not Related | 37.1 | 25.5 |
| AEs by Severity | | |
| Mild | 22.0 | 10.6 |
| Moderate | 16.7 | 19.1 |
| Severe | 3.0 | 4.3 |
| Life Threatening | 0.0 | 0.0 |

TABLE 30
Follow-on Study: GI Adverse Events

Incidence % (N)

| | HZT-501 TID | Ibuprofen 800 mg TID |
|---|---|---|
| Adverse Event | (N = 132) | (N = 47) |
| Any GI Event | 8.3 (11) | 4.3 (2)* |
| Dyspepsia | 2.3 (3) | 0.0 (0) |
| Abdominal Pain Upper | 1.5 (2) | 0.0 (0) |
| Abdominal Tenderness | 0.0 (0) | 2.1 (0) |
| Nausea | 1.5 (2) | 0.0 (0) |
| Diarrhea | 0.0 (0) | 2.1 (1) |
| Constipation | 1.5 (2) | 0.0 (0) |
| Withdrawals due to GI | 0.8 (1) | 0.0 (0) |

*P = 0.412

TABLE 31
Follow-on Study: Commonly Observed Adverse Events

| | Incidence % (N) | |
|---|---|---|
| | HZT-501 TID | Ibuprofen 800 mg TID |
| Adverse Event | (N = 132) | (N = 47) |
| General | 0.8(1) | 4.3(2) |
| Disorders/Administrative Site | | |
| Conditions | | |
| Infections/Infestations | 18.2(24) | 14.9(7) |
| Injury, Poisoning, Procedural | 3.8(5) | 4.3(2) |
| Complications | | |
| Metabolism and Nutrition | 2.3(3) | 8.5(4)* |
| Disorders | | |
| Musculoskeletal/connective | 9.8(13) | 2.1(1) |
| Tissue Disorders | | |
| Nervous System Disorders | 4.5(6) | 4.3(2) |
| Respiratory, Thoracic and | 3.0(4) | 2.1(1) |
| Mediastinal Disorders | | |

*P = 0.027

TABLE 32
Follow-on Study: Adverse Events of Interest

| | Incidence %(N) | |
|---|---|---|
| | HZT-501 TID | Ibuprofen 800 mg TID |
| Adverse Event | (N = 132) | (N = 47) |
| Respiratory, Thoracic and | 3.0(4) | 2.1(1) |
| Mediastinal Disorders | | |
| Nervous System Disorder | 4.5(6) | 4.3(2) |
| Hepatobiliary Disorders | 0.8(1) | 2.1(1) |
| Hypertension | 3.0(4) | 0.0(0) |

TABLE 33
Follow-on Study: Hepatic Effects

| | Number of Subjects | |
|---|---|---|
| | HZT-501 TID | Ibuprofen 800 mg TID |
| Shift from Normal to High* | (N = 132) | (N = 47) |

AST

| | | |
|---|---|---|
| 14 weeks | 0 | 0 |
| 28 weeks | 0 | 0 |

ALT

| | | |
|---|---|---|
| 14 weeks | 0 | 0 |
| 28 weeks | 0 | 0 |

AST = aspartate aminotransferase; ALT = alanine aminotransferase
*from Day 0 to the study week indicated.

TABLE 34
Follow-on Study: Serious Adverse Events (“SAEs”) and Deaths

| | Incidence %(N) | |
|---|---|---|
| | HZT-501 TID | Ibuprofen 800 mg TID |
| | (N = 132) | (N = 47) |
| Total SAEs | 3.0(4) | 4.3(2) |
| SAEs leading to discontinuation | 0.8(1) | 0.0(0) |
| Deaths | 0.0(0) | 0.0(0) |

TABLE 35
Follow-on Study: Treatment-emergent Adverse Events occurring
in 1% or greater of the safety follow-on population

| | HZT-501 | Ibuprofen | Total | |
|---|---|---|---|---|
| System Organ Class | N = 132 | N = 47 | N = 179 | |
| Preferred Term | % (n) | % n) | % (n) | P-Value* |
| Total Subjects With at Least One TEAE | 68.2 (90) | 68.1 (32) | 68.2 (122) | 0.9879 |
| Blood and lymphatic system | 2.3 (3) | 2.1 (1) | 2.2 (4) | 0.9432 |
| disorders | | | | |
| Anaemia | 2.3 (3) | 2.1 (1) | 2.2 (4) | 0.9432 |
| Ear and labyrinth disorders | 2.3 (3) | 2.1 (1) | 2.2 (4) | 0.9149 |
| Tinnitus | 0.0 (0) | 2.1 (1) | 0.6 (1) | 0.1031 |
| Eye disorders | 2.3 (3) | 4.3 (2) | 2.8 (5) | 0.4746 |
| Cataract | 0.0 (0) | 2.1 (1) | 0.6 (1) | 0.0767 |
| Conjunctivitis | 0.0 (0) | 2.1 (1) | 0.6 (1) | 0.1031 |
| Gastrointestinal disorders | 26.5 (35) | 23.4 (11) | 25.7 (46) | 0.7029 |
| Diarrhoea | 3.8 (5) | 8.5 (4) | 5.0 (9) | 0.1841 |
| Dyspepsia | 6.1 (8) | 2.1 (1) | 5.0 (9) | 0.3074 |
| Constipation | 3.8 (5) | 4.3 (2) | 3.9 (7) | 0.9407 |
| Nausea | 3.8 (5) | 4.3 (2) | 3.9 (7) | 0.8747 |
| Abdominal pain upper | 2.3 (3) | 2.1 (1) | 2.2 (4) | 0.9716 |
| Gastrooesophageal reflux disease | 2.3 (3) | 2.1 (1) | 2.2 (4) | 0.9432 |
| Abdominal distension | 2.3 (3) | 0.0 (0) | 1.7 (3) | 0.2991 |
| Abdominal tenderness | 1.5 (2) | 2.1 (1) | 1.7 (3) | 0.8146 |
| Stomach discomfort | 2.3 (3) | 0.0 (0) | 1.7 (3) | 0.2837 |
| Vomiting | 0.8 (1) | 4.3 (2) | 1.7 (3) | 0.1233 |
| Abdominal discomfort | 0.8 (1) | 2.1 (1) | 1.1 (2) | 0.3902 |
| Abdominal pain | 0.8 (1) | 2.1 (1) | 1.1 (2) | 0.4705 |
| Flatulence | 0.8 (1) | 2.1 (1) | 1.1 (2) | 0.4705 |
| Gastritis | 0.8 (1) | 2.1 (1) | 1.1 (2) | 0.4331 |
| Toothache | 0.8 (1) | 2.1 (1) | 1.1 (2) | 0.4705 |
| Intestinal spasm | 0.0 (0) | 2.1 (1) | 0.6 (1) | 0.1031 |
| Oesophageal stenosis | 0.0 (0) | 2.1 (1) | 0.6 (1) | 0.1031 |
| General disorders and administration | 4.5 (6) | 6.4 (3) | 5.0 (9) | 0.6245 |
| site conditions | | | | |
| Oedema peripheral | 1.5 (2) | 6.4 (3) | 2.8 (5) | 0.0890 |
| Non-cardiac chest pain | 1.5 (2) | 2.1 (1) | 1.7 (3) | 0.8146 |
| Gait disturbance | 0.0 (0) | 2.1 (1) | 0.6 (1) | 0.1031 |
| Hepatobiliary disorders | 0.8 (1) | 2.1 (1) | 1.1 (2) | 0.4705 |
| Cholelithiasis | 0.0 (0) | 2.1 (1) | 0.6 (1) | 0.1031 |
| Immune system disorders | 1.5 (2) | 0.0 (0) | 1.1 (2) | 0.4044 |
| Infections and infestations | 34.8 (46) | 36.2 (17) | 35.2 (63) | 0.8784 |
| Upper respiratory tract infection | 6.1 (8) | 6.4 (3) | 6.1 (11) | 0.9339 |
| Influenza | 6.1 (8) | 2.1 (1) | 5.0 (9) | 0.2906 |
| Gastroenteritis viral | 3.8 (5) | 4.3 (2) | 3.9 (7) | 0.8971 |
| Sinusitis | 4.5 (6) | 2.1 (1) | 3.9 (7) | 0.4865 |
| Urinary tract infection | 4.5 (6) | 0.0 (0) | 3.4 (6) | 0.1244 |
| Bronchitis | 3.0 (4) | 2.1 (1) | 2.8 (5) | 0.7544 |
| Nasopharyngitis | 3.0 (4) | 2.1 (1) | 2.8 (5) | 0.7314 |
| Gastroenteritis | 3.0 (4) | 0.0 (0) | 2.2 (4) | 0.2354 |
| Viral upper respiratory tract | 2.3 (3) | 2.1 (1) | 2.2 (4) | 0.9149 |
| infection | | | | |
| Bronchitis acute | 0.8 (1) | 2.1 (1) | 1.1 (2) | 0.4705 |
| Ear infection | 0.8 (1) | 2.1 (1) | 1.1 (2) | 0.4705 |
| Laryngitis | 0.0 (0) | 4.3 (2) | 1.1 (2) | 0.0163 |
| Lower respiratory tract infection | 0.0 (0) | 4.3 (2) | 1.1 (2) | 0.0163 |
| Otitis media | 1.5 (2) | 0.0 (0) | 1.1 (2) | 0.4205 |
| Pneumonia | 0.8 (1) | 2.1 (1) | 1.1 (2) | 0.4331 |
| Tooth abscess | 1.5 (2) | 0.0 (0) | 1.1 (2) | 0.3835 |
| Tooth infection | 0.8 (1) | 2.1 (1) | 1.1 (2) | 0.3902 |
| Folliculitis | 0.0 (0) | 2.1 (1) | 0.6 (1) | 0.1031 |
| Viral infection | 0.0 (0) | 2.1 (1) | 0.6 (1) | 0.0767 |
| Injury, poisoning and procedural | 7.6 (10) | 17.0 (8) | 10.1 (18) | 0.0694 |
| complications | | | | |
| Traumatic brain injury | 1.5 (2) | 2.1 (1) | 1.7 (3) | 0.8146 |
| Animal bite | 1.5 (2) | 0.0 (0) | 1.1 (2) | 0.3835 |
| Foot fracture | 0.8 (1) | 2.1 (1) | 1.1 (2) | 0.4331 |
| Rib fracture | 0.0 (0) | 4.3 (2) | 1.1 (2) | 0.0206 |
| Skin laceration | 0.8 (1) | 2.1 (1) | 1.1 (2) | 0.4705 |
| Tendon injury | 1.5 (2) | 0.0 (0) | 1.1 (2) | 0.3835 |
| Accidental overdose | 0.0 (0) | 2.1 (1) | 0.6 (1) | 0.1031 |
| Contusion | 0.0 (0) | 2.1 (1) | 0.6 (1) | 0.0767 |
| Fall | 0.0 (0) | 2.1 (1) | 0.6 (1) | 0.0767 |
| Joint injury | 0.0 (0) | 2.1 (1) | 0.6 (1) | 0.1031 |
| Joint sprain | 0.0 (0) | 2.1 (1) | 0.6 (1) | 0.0767 |
| Limb crushing injury | 0.0 (0) | 2.1 (1) | 0.6 (1) | 0.1031 |
| Neck injury | 0.0 (0) | 2.1 (1) | 0.6 (1) | 0.1031 |
| Road traffic accident | 0.0 (0) | 2.1 (1) | 0.6 (1) | 0.1031 |
| Investigations | 3.8 (5) | 0.0 (0) | 2.8 (5) | 0.1860 |
| Blood creatinine increased | 1.5 (2) | 0.0 (0) | 1.1 (2) | 0.4205 |
| Blood pressure increased | 1.5 (2) | 0.0 (0) | 1.1 (2) | 0.4044 |
| Metabolism and nutrition disorders | 3.0 (4) | 8.5 (4) | 4.5 (8) | 0.1382 |
| Diabetes mellitus | 2.3 (3) | 2.1 (1) | 2.2 (4) | 0.9149 |
| Diabetes mellitus non-insulin- | 0.0 (0) | 2.1 (1) | 0.6 (1) | 0.1031 |
| dependent | | | | |
| Hypercholesterolaemia | 0.0 (0) | 2.1 (1) | 0.6 (1) | 0.1031 |
| Hyperglycaemia | 0.0 (0) | 2.1 (1) | 0.6 (1) | 0.1031 |
| Hyperlipidaemia | 0.0 (0) | 2.1 (1) | 0.6 (1) | 0.1031 |
| Hyperosmolar state | 0.0 (0) | 2.1 (1) | 0.6 (1) | 0.1031 |
| Musculoskeletal and connective | 12.9 (17) | 12.8 (6) | 12.8 (23) | 0.9720 |
| tissue disorders | | | | |
| Arthralgia | 4.5 (6) | 4.3 (2) | 4.5 (8) | 0.9188 |
| Back pain | 3.8 (5) | 0.0 (0) | 2.8 (5) | 0.1722 |
| Musculoskeletal pain | 1.5 (2) | 4.3 (2) | 2.2 (4) | 0.2418 |
| Muscle spasms | 2.3 (3) | 0.0 (0) | 1.7 (3) | 0.2837 |
| Neck pain | 1.5 (2) | 2.1 (1) | 1.7 (3) | 0.7457 |
| Tendonitis | 1.5 (2) | 2.1 (1) | 1.7 (3) | 0.7812 |
| Joint swelling | 0.8 (1) | 2.1 (1) | 1.1 (2) | 0.4331 |
| Knee deformity | 0.0 (0) | 2.1 (1) | 0.6 (1) | 0.1031 |
| Plantar fasciitis | 0.0 (0) | 2.1 (1) | 0.6 (1) | 0.1031 |
| Neoplasms benign, malignant and | 1.5 (2) | 0.0 (0) | 1.1 (2) | 0.3835 |
| unspecified (incl. cysts and polyps) | | | | |
| Nervous system disorders | 10.6 (14) | 6.4 (3) | 9.5 (17) | 0.3802 |
| Dizziness | 2.3 (3) | 2.1 (1) | 2.2 (4) | 0.9149 |
| Carpal tunnel syndrome | 2.3 (3) | 0.0 (0) | 1.7 (3) | 0.3113 |
| Headache | 2.3 (3) | 0.0 (0) | 1.7 (3) | 0.2991 |
| Migraine | 0.8 (1) | 2.1 (1) | 1.1 (2) | 0.4705 |
| Nerve compression | 0.0 (0) | 2.1 (1) | 0.6 (1) | 0.1031 |
| Psychiatric disorders | 1.5 (2) | 4.3 (2) | 2.2 (4) | 0.2646 |
| Anxiety | 0.8 (1) | 2.1 (1) | 1.1 (2) | 0.4331 |
| Mood swings | 0.0 (0) | 2.1 (1) | 0.6 (1) | 0.0767 |
| Renal and urinary disorders | 1.5 (2) | 0.0 (0) | 1.1 (2) | 0.4044 |
| Respiratory, thoracic and mediastinal | 9.8 (13) | 4.3 (2) | 8.4 (15) | 0.2421 |
| disorders | | | | |
| Cough | 3.0 (4) | 0.0 (0) | 2.2 (4) | 0.2257 |
| Pharyngolaryngeal pain | 1.5 (2) | 2.1 (1) | 1.7 (3) | 0.7812 |
| Asthma | 1.5 (2) | 0.0 (0) | 1.1 (2) | 0.4205 |
| Throat irritation | 0.8 (1) | 2.1 (1) | 1.1 (2) | 0.4705 |
| Wheezing | 1.5 (2) | 0.0 (0) | 1.1 (2) | 0.3835 |
| Skin and subcutaneous tissue | 6.8 (9) | 6.4 (3) | 6.7 (12) | 0.8994 |
| disorders | | | | |
| Eczema | 1.5 (2) | 2.1 (1) | 1.7 (3) | 0.8146 |
| Dermatitis contact | 0.8 (1) | 2.1 (1) | 1.1 (2) | 0.4331 |
| Swelling face | 1.5 (2) | 0.0 (0) | 1.1 (2) | 0.4044 |
| Dermatitis | 0.0 (0) | 2.1 (1) | 0.6 (1) | 0.1031 |
| Urticaria | 0.0 (0) | 2.1 (1) | 0.6 (1) | 0.1031 |
| Vascular disorders | 9.1 (12) | 2.1 (1) | 7.3 (13) | 0.1046 |
| Hypertension | 8.3 (11) | 2.1 (1) | 6.7 (12) | 0.1275 |

TEAE = treatment-emergent adverse event
All adverse events were coded using the MedDRA dictionary (Version 9.1). A subject with multiple events per System Organ Class or per Preferred Term category is counted only once per subject.
*P-Value is calculated is from a Cochran-Mantel-Haenszel test for adverse events occurring in ≧1% of subjects for the Integrated Data.

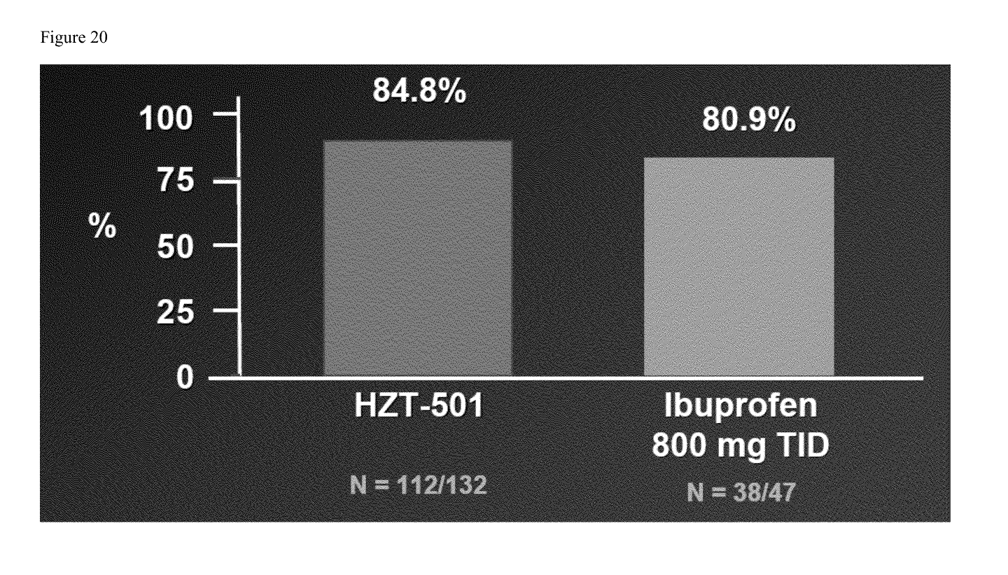

Percentage of subjects who withdraw from the study due to adverse events is shown in FIG. 20.

### C. Summary

Based on the foregoing results, it is concluded that:

(1) The safety profile of HZT-501 is similar to that of ibuprofen;

(2) HZT-501 did not cause more serious adverse events than ibuprofen; and

(3) no deaths occurred in either HZT-501 or ibuprofen follow-on study.

Of the 1022 subjects in clinical studies of HZT-501, 15% (213 subjects) used low-dose aspirin and the results were consistent with the overall findings of the study. In these clinical studies 16% of subjects who used low-dose aspirin who were treated with HZT-501 developed an upper gastrointestinal ulcer compared to 35% of those subjects who received only ibuprofen.

The clinical trials primarily enrolled subjects less than 65 years without a prior history of gastrointestinal ulcer. Of the 1022 subjects in clinical studies of HZT-501, 18% (249 subjects) were 65 years of age or older. In these clinical studies, 23% of subjects 65 years of age and older who were treated with HZT-501 developed an upper gastrointestinal ulcer compared to 27% of those subjects who received only ibuprofen.

Of the 1022 subjects in clinical studies of HZT-501, 6% had a prior history of gastrointestinal ulcer. In these clinical studies, 25% of subjects with a prior history of gastrointestinal ulcer who were treated with HZT-501 developed an upper gastrointestinal ulcer compared to 24% of those subjects who received only ibuprofen.

It is understood that the examples and embodiments described herein are for illustrative purposes only and that various modifications or changes in light thereof will be suggested to persons skilled in the art and are to be included within the spirit and purview of this application and scope of the appended claims.

1.-43. (canceled)

44. A pharmaceutical composition having a bilayer architecture comprising:
a first layer comprising famotidine and further comprising hypromellose (hydroxypropylmethylcellulose), and
a second layer comprising ibuprofen,
wherein the pharmaceutical composition is suitable for three times per day (TID) administration, and
wherein both the famotidine and ibuprofen are formulated for immediate release at about the same time.

45. The pharmaceutical composition of claim 44, wherein the pharmaceutical composition comprises about 24 mg to about 28 mg famotidine.

46. The pharmaceutical composition of claim 44, wherein the pharmaceutical composition comprises about 750 mg to about 850 mg ibuprofen.

47. The pharmaceutical composition of claim 44, wherein the first layer further comprises at least one lubricant.

48. The pharmaceutical composition of claim 47, wherein the at least one lubricant is magnesium stearate.

49. The pharmaceutical composition of claim 44, wherein the first layer further comprises at least one binder other than hypromellose (hydroxypropylmethylcellulose).

50. The pharmaceutical composition of claim 49, wherein the at least one binder is microcrystalline cellulose.

51. The pharmaceutical composition of claim 44, wherein the first layer further comprises at least one glidant.

52. The pharmaceutical composition of claim 51, wherein the at least one glidant is colloidal silicon dioxide.

53. The pharmaceutical composition of claim 44, wherein the first layer comprises famotidine, magnesium stearate, microcrystalline cellulose, hypromellose, and colloidal silicon dioxide.

54. The pharmaceutical composition of claim 44, wherein the second layer further comprises at least one binder.

55. The pharmaceutical composition of claim 54, wherein the at least one binder is microcrystalline cellulose.

56. The pharmaceutical composition of claim 55, wherein the second layer comprises at least one binder other than microcrystalline cellulose.

57. The pharmaceutical composition of claim 56, wherein the at least one binder other than microcrystalline cellulose is chosen from Klucel EXF hydroxypropyl cellulose, propylene glycol, Starch 1500, Lubritab, Kollidon VA 64 vinylpyrrolidone-vinyl acetate copolymer, PVP K 30 polyvinyl pyrrolidone, sodium stearyl fumarate, and stearic acid.

58. The pharmaceutical composition of claim 56, wherein at least one binder other than microcrystalline cellulose is hypromellose (hydroxypropylmethylcellulose).

59. The pharmaceutical composition of claim 44, wherein the second layer further comprises at least one lubricant.

60. The pharmaceutical composition of claim 59, wherein the at least one lubricant is sodium stearyl fumarate.

61. The pharmaceutical composition of claim 44, wherein the second layer further comprises at least one glidant.

62. The pharmaceutical composition of claim 61, wherein the at least one glidant is colloidal silicon dioxide.

63. The pharmaceutical composition of claim 44, wherein the second layer further comprises at least one disintegrant.

64. The pharmaceutical composition of claim 63, wherein at least one disintegrant is croscarmellose sodium.

65. The pharmaceutical composition of claim 44, wherein the second layer comprises ibuprofen, microcrystalline cellulose, sodium stearyl fumarate, colloidal silicon dioxide, hypromellose, and croscarmellose sodium.

66. The pharmaceutical composition of claim 53, wherein the second layer comprises ibuprofen, microcrystalline cellulose, sodium stearyl fumarate, colloidal silicon dioxide, hypromellose, and croscarmellose sodium.

67. The pharmaceutical composition of claim 44, wherein the first layer and the second layer are separated by a barrier layer.

68. A pharmaceutical composition having a trilayer architecture comprising
a first layer comprising a therapeutically effective amount of famotidine,
a second layer comprising ibuprofen, and
a third layer comprising ibuprofen,
wherein the first layer is adjacent to a first side of the second layer, and the third layer is adjacent to a second side of the first layer,
wherein the total amount of ibuprofen in the pharmaceutical composition is a therapeutically effective amount,
wherein the pharmaceutical composition is suitable for three times per day (TID) administration, and
wherein both the famotidine and ibuprofen are formulated for immediate release at about the same time.

69. The pharmaceutical composition of claim 68, wherein the first layer and the second layer are separated by a first barrier layer, and the first layer and the third layer are separated by a second barrier layer.

70. The pharmaceutical composition of claim 68, wherein the first barrier layer is the same, both in amount and content, as the second barrier layer.

71. The pharmaceutical composition of claim 68, wherein the first barrier layer is different, either in amount and/or content, from the second barrier layer.

72. A pharmaceutical composition comprising:
a first compartment comprising
a therapeutically effective amount of famotidine;
from about 42 mg to about 46 mg of microcrystalline cellulose;
from about 10 mg to about 19 mg of at least one binder other than microcrystalline cellulose; and
from about 0.9 mg to about 1.9 mg of at least one lubricant, and
a second compartment comprising
a therapeutically effective amount of ibuprofen;
from about 200 to about 250 mg of at least one binder; and
from about 2.5 mg to about 3.5 mg of at least one lubricant,
wherein said first compartment is separated from said second compartment.

73. A method for method for reducing the risk of developing ibuprofen-induced ulcers in a human patient requiring ibuprofen for an ibuprofen-responsive condition, said method comprising:
administering to the human patient a first dose of famotidine,
administering to the human patient a second dose of famotidine, and
administering to the human patient a third dose of famotidine, and
wherein for each administration, the famotidine is administered as a pharmaceutical composition of claim 44.
